(19) United States
(12) Patent Application Publication
(10) Pub. No.: US 2026/0166242 A1
(43) Pub. Date: Jun. 18, 2026

(54) DRUG DELIVERY DEVICE WITH ELECTRONICS

(71) Applicant: Norton (Waterford) Limited, Waterford (IE)

(72) Inventors: Niall Thompson, Waterford (IE); Declan Walsh, Waterford (IE); Paul Prendergast, Waterford (IE); Daniel Buck, Waterford (IE)

(73) Assignee: Norton (Waterford) Limited, Waterford (IE)

(21) Appl. No.: 18/711,763

(22) PCT Filed: Nov. 24, 2022
(86) PCT No.: PCT/EP2022/083189

§ 371 Date: May 20, 2024

Related U.S. Application Data
(60) Provisional application No. 63/264,521, filed on Nov. 24, 2021.

(51) Int. Cl. A61M 15/00

(52) U.S. Cl. CPC A61M 15/009; A61M 15/0026; A61M 15/0078; A61M 15/0091; A61M 2205/3334; A61M 2205/52; A61M 2205/8212

(57) ABSTRACT

An inhalation device may include a canister elongate along a longitudinal axis, the canister containing medicament. The inhalation device may also include a movable inner housing that is configured to move with the canister when the medication canister is moved to administer a dose of medicament. The inhalation device may include an electronics module comprising a processor, a power supply, a sensor, and a plurality of contact pads. Further, the inhalation device may include a switch contact affixed to the moveable inner housing. In response to movement of the movable inner housing, the switch contact may be configured to move from a first position where the switch contact is not contacting the plurality contact pads to a second position where the switch contact is contacting at least one of the plurality of contact pads to change a power state of one or more of the processor and sensor.

### CROSS-REFERENCE TO RELATED APPLICATIONS

This application claims the benefit of U.S. Provisional Patent Application No. 63/264,521, filed Nov. 24, 2021, the contents of which are incorporated herein by reference in its entirety.

### BACKGROUND

Drug delivery devices facilitate the delivery of medication into a patient's body via various routes of administration. Typical routes of administration include oral, topical, sublingual inhalation, injection, and the like. The devices may be used to deliver medications for the treatment various diseases, ailments, and medical conditions. Inhalation devices, for example, may be used to treat asthma, chronic obstructive pulmonary disease (COPD) and cystic fibrosis (CF). While drug delivery devices are designed to deliver an appropriate dose of medication to a patient as part of a therapeutic treatment, the effectiveness of a particular treatment may be influenced by non-physiological factors, such as the patient's adherence and compliance.

In the context of a drug therapy, adherence may refer to the degree to which a patient is following a prescribed dosing regimen. For example, if the patient's prescription calls for two doses each day, and the patient is taking two doses per day, the patient may be considered 100% adherent. If the patient is only taking one dose per day, he or she may be deemed only 50% adherent. In the latter case, the patient may not be receiving the treatment prescribed by his or her doctor, which may negatively affect the efficacy of the therapeutic treatment.

Compliance may refer to a patient's technique when using a particular drug delivery device. If the patient is using the device in a manner that is recommended by a doctor or by a manufacturer, the device is likely to deliver the desired dose of medication and the patient may be deemed compliant. However, if the device is not being used properly during drug administration, the device's ability to deliver a proper dose of medication may be compromised. As such, the patient may be deemed non-compliant. In the case of an inhalation device, for example, the patient may need to achieve a minimum inspiratory effort to ensure a full dose of medication is delivered from the device into the patient's lungs. For some patients, such as children and the elderly, meeting the requirements for full compliance may be difficult due to physical limitations, such as limited lung function. Accordingly, like adherence, failing to achieve full compliance may reduce the effectiveness of a prescribed treatment.

A patient's ability to achieve full compliance may be further complicated by certain physical properties of the medication. For example, some respiratory medications may consist of fine particles and/or may lack any odor or taste. Thus, a patient using an inhalation device may not be able to correct a non-compliant use because he or she may not be able to immediately detect or sense that medication is being inhaled and/or know whether the amount of inhaled medication complies with the prescription.

### SUMMARY

An inhaler may include a canister, a movable inner housing, an electronics module, and a switch contact. The canister may be elongate along a longitudinal axis. The canister may contain medicament. The movable inner housing may be configured to move with the canister when the medication canister is moved to administer a dose of medicament. For example, the movable inner housing may cause the canister to move to administer a dose of medicament. The electronics module may include a processor, a power supply, a sensor, and a plurality of contact pads. The switch contact may be affixed to the moveable inner housing. In response to movement of the movable inner housing, the switch contact may be configured to move from a first position where the switch contact is not contacting the plurality contact pads to a second position where the switch contact is contacting at least one of the plurality of contact pads to change a power state of one or more of the processor and sensor. The sensor may include a pressure sensor, such as a barometric pressure sensor, and/or an acoustic sensor.

The movable inner housing may substantially surround the canister (e.g., entirely surround the canister). The switch contact may be affixed to an outer surface of the moveable inner housing. The movable inner housing may be configured to be in a first configuration when the canister is in a first position, a second configuration spaced from the first configuration along the longitudinal axis when the canister is in a second position, and a third configuration spaced from the first and second configurations along the longitudinal axis when the canister is in a third position. The canister may be configured to administer a dose of medicament when moved from the second position to the third position. The canister may be configured to move from the first position to the second position in response to a movement of a mouthpiece cover of the inhaler to expose a mouthpiece of the inhaler. In some examples, the first configuration may be a rest configuration in which a metering valve of the canister is in a refill position, the second configuration may be a prepared configuration in which the medication canister is actuatable by user inhalation induced airflow, and the third configuration may be an actuation configuration in which the metering valve is in a dose delivery position. In some instances, the medication canister cannot be actuated to administer a dose of medicament when the movable inner housing in the first configuration.

The movable inner housing may include a force holding unit configured to maintain a force for actuating the canister until a user inhales through the mouthpiece of the inhaler. For example, the force holding unit may include a compression spring, a lower cap that engages the canister, a diaphragm attached to an upper surface of the lower cap, and a pivotally mounted flap valve for selectively sealing a valve port located in the diaphragm. For example, an inhalation by a user may cause air to pivot the flap valve, which opens a valve channel in the diaphragm allowing passage of the air into a volume between the diaphragm and the lower cap, which may produce expansion of the compression spring and a downward motion of the canister, and actuation of a metering valve of the canister to release a measured dose through into a mouthpiece of the inhaler. The inhaler may include a main housing that encloses the canister, the electronics module, and the movable inner housing.

The plurality of contact pads may include a first contact pad, a second contact pad, and a third contact pad. The switch contact may include a first prong that is configured to contact either the first contact pad or the third contact pad, and a second prong that is configured to contact the second contact pad. In some examples, the first prong may be configured to bend independently of the second prong. The first prong may include a nub configured to contact the first contact pad, and the second prong may include a nub configured to contact the second contact pad. When the switch contact is in the first position, the first and second prongs may be configured to not be in contact with any of the first, second, or third contact pads. In some examples, the first, second, and third contact pads may be substantially rectangularly shaped.

When the switch contact is in the second position, the first prong may be configured to be in contact with the first contact pad, and the second prong may be configured to be in contact with the second contact pad. Further, in some instance, when the switch contact is in the second position, a metering valve of the canister may be in a refill position (e.g., the same position that the metering valve is in when the switch contact is in the first position) or a pre-actuation position (e.g., where the metering valve is slightly depressed, but not enough to cause a dose of medicament to be metered from the canister). When the switch contact is in a third position, the first prong may be configured to be in contact with the third contact pad, the second prong is configured to be in contact with (e.g., remain in contact with) the second contact pad, and the metering valve of the canister is configured to be in a dose delivery position. In some examples, the second prong of the switch contact may be in continuous contact with the second contact pad as the switch contact moves from the second position to the third position. When the switch contact moves into the second position, the processor may be configured to record a mouthpiece cover opening event. When the switch contact moves into the third position, the processor may be configured to record a canister actuation event.

The first contact pad may be located above the third contact pad on a printed circuit board (PCB) and adjacent the second contact pad, and the third contact pad may be located below the first contact pad on the PCB and adjacent the second contact pad. In some examples, the first, second, and third contact pads may each define a respective longitudinal length, where the longitudinal length of the second contact pad is substantially equal to a length defined between an upper, transverse edge of the first contact pad and a lower, transverse edge of the third contact pad. The longitudinal length of the second contact pad may be greater than the longitudinal lengths of the first and third contact pads combined. Each of the contact pads may include a planar electrically conductive strip attached to a printed circuit board (PCB) of the electronics module.

The plurality of contact pads may include a first contact pad, a second contact pad, and a third contact pad. In some examples, the switch contact may include a single prong that is configured to contact the first contact pad and the second contact pad simultaneously, and configured to contact the second contact pad and the third contact pad simultaneously. When the switch contact is in the first position, the single prong may be configured to not be in contact with any of the first, second, or third contact pads, and when the switch contact is in the second position, the single prong may be configured to be in contact with the first contact pad and the second contact pad. Further, when the switch contact is in the second position, the metering valve of the canister may be in a refill position (e.g., the same position that the metering valve is in when the switch contact is in the first position) or a pre-actuation position (e.g., where the metering valve is slightly depressed, but not enough to cause a dose of medicament to be metered from the canister). When the switch contact is in a third position, the single prong may be configured to be in contact with the second contact pad and the third contact pad, and the metering valve of the canister is configured to be in a dose delivery position. The single prong of the switch contact may be in continuous contact with the second contact pad as the switch contact moves from the second position to the third position. When the switch contact moves into the second position, the processor may be configured to record a mouthpiece cover opening event, and when the switch contact moves into the third position, the processor may be configured to record a canister actuation event.

When the switch contact is in the first position, the processor may be configured to be in an off state or a sleep state and the pressure sensor is configured to be in an off state. When the switch contact is in the second position, the processor may be configured to be in an active state and the pressure sensor is configured to be in an active state.

The electronics module may include a printed-circuit board (PCB) that defines a first portion and a second portion. The first portion may include the processor, the power supply, and the sensor, and the second portion may include the contact pads. In some examples, the first portion may be oriented at a substantially perpendicular angle with respect to the second portion. The first portion of the PCB may reside between a top surface of the medication canister and a top, interior surface of a main housing of the inhaler, and the second portion of the PCB may extend along an inner, sidewall of the main housing.

An inhaler may include a canister and an electronics module. The canister may include medicament and a metering valve. The electronics module may include a processor, a power supply, and a sensor. The inhaler may be configured to be in a rest configuration in which the metering valve of the canister is in a refill position, a prepared configuration in which the canister is actuatable by patient inhalation induced airflow, and an actuation configuration in which the metering valve is in a dose delivery position. The processor may be configured to record a mouthpiece cover opening event in response to the inhaler moving from the rest configuration to the prepared configuration, record a canister actuation event in response to the inhaler moving from the prepared configuration to the actuation configuration, and record one or more signals from the sensor indicative of a user's inhalation when the inhaler is in the actuation configuration (e.g., record one or more signals from the sensor indicative of a user's inhalation when the inhaler is in the prepared configuration and the actuation configuration).

The electronics module may also include a communication circuit, and wherein the processor is configured to cause the communication circuit to wirelessly transmit, to an external device, the mouthpiece cover opening event, the canister actuation event, and the one or more signals from the sensor indicative of the user's inhalation.

The sensor may include a pressure sensor, and the one or more signals comprises pressure measurements recorded by the pressure sensor, and/or may include an acoustic sensor, and the one or more signals comprises acoustic measurements recorded by the acoustic sensor. The processor may be configured to record the one or more signals from the sensor indicative of the user's inhalation in response to the inhaler moving from the prepared configuration to the actuation configuration.

The inhaler may be part of a system that includes a computer-readable storage medium comprising executable instructions (e.g., a mobile application) that resides on an external device that comprises a processor. The computer-readable storage medium may be configured to cause the processor of the external device to receive the mouthpiece cover opening event, the canister actuation event, and the one or more signals from the sensor indicative of the user's inhalation. The computer-readable storage medium may be configured to cause the processor of the external device to generate an error event when the one or more signals from the sensor indicative of the user's inhalation indicate a measurement below a threshold. In response to the error event, the computer-readable storage medium may be configured to cause the processor of the external device to generate a graphical user interface (GUI) that indicates that a misuse event occurred that caused a dose of medicament to be dispensed without a sufficient inhalation. The computer-readable storage medium may be configured to cause the processor of the external device to generate a multiple inhalation event in response to more than one user inhalation being associated with a single canister actuation event. The computer-readable storage medium may be configured to cause the processor of the external device to determine that a user inhalation occurred during a canister actuation event based on the one or more signals from the sensor indicative of the user's inhalation and one or more thresholds. The computer-readable storage medium may be configured to cause the processor of the external device to record an inhalation event that includes the mouthpiece cover opening event, the canister actuation event, and the one or more signals from the sensor indicative of the user's inhalation, and send the inhalation event to a remote server.

The computer-readable storage medium may be configured to cause the processor of the external device to receive the mouthpiece cover opening event, the canister actuation event, and the one or more signals from the sensor indicative of the user's inhalation. The computer-readable storage medium may be configured to cause the processor of the external device to generate an error event in response to receiving the mouthpiece cover opening event, the canister actuation event, and the one or more signals from the sensor indicative of the user's inhalation from a single usage event when the one or more signals from the sensor indicative of the user's inhalation associated with the canister actuation event indicate a measurement below a threshold.

In some examples, the system may include a remote server that is configured to aggregate a plurality of different inhalation events for the user, and a plurality of different inhalation events for a plurality of different users.

An inhaler may include a canister (e.g., including medicament), and an electronics module that includes a processor, a power supply, a sensor, a first contact pad, and a second contact pad. The inhaler may include a switch contact comprising a first prong and a second prong. The processor and/or pressure sensor may be configured to change power states in response to the first prong contacting the first contact pad while the second prong contacts the second contact pad. The first prong may be configured to bend independently of the second prong.

An inhaler may include a main housing defining a mouthpiece, and a canister comprising medicament residing within the main housing. The inhaler may include a printed-circuit board (PCB) that includes a processor, a power supply, and a sensor. The PCB may reside between a top surface of the canister and a top, interior surface of a main housing of the inhaler, and wherein the sensor is located on a bottom surface of the PCB. In response to changing power states, the processor may be configured to receive one or more measurements from the sensor. The PCB may define a top surface located in closer proximity to the top, interior surface of the main housing of the inhaler than the bottom surface of the PCB. The PCB defines a front side that is in closer proximity to an opening of the mouthpiece cover that a back side of the PCB. In some examples, the sensor may be located on the bottom surface of the PCB at the front side of the PCB. In some examples, the sensor may be located on the bottom surface of the PCB at the back side of the PCB. A top surface of the main housing may define one or more air vents. The plurality of air vents may include a first air vent located adjacent an upper, left side of the PCB and a second air vent located adjacent an upper, right side of the PCB. The power supply may be located on the top surface of the PCB, and the processor and the sensor may be located on the bottom surface of the PCB. The bottom surface of the PCB may include an antenna for a communication circuit. The main housing may define a plurality of longitudinal ribs. The PCB may define a first portion that comprises the processor, the power supply, a communication circuit, and the sensor, and a second portion that comprises a plurality of contact pads. The first portion may be oriented at a substantially perpendicular angle with respect to the second portion. The PCB may be configured such that, when the contact pads are contacts by a switch contact, the PCB is configured to cause the sensor to change from a first power state to a second power state.

### BRIEF DESCRIPTION OF THE DRAWINGS

FIG. 1 is a front perspective view of an example inhalation device.

FIG. 2 is a front perspective view of the example inhalation device of FIG. 1 with the mouthpiece cover in the open position.

FIG. 3 is a partially exploded view of the example inhalation device of FIG. 1.

FIG. 4 is a partially exploded view of the example inhalation device of FIG. 1.

FIG. 5 is a partially exploded view of a movable inner housing of the example inhalation device of FIG. 1.

FIG. 6 is a perspective view of the top portion of the main housing of the inhalation device of FIG. 1.

FIG. 7 is a partially exploded view of the top portion of the main housing and an electronics module of the inhalation device.

FIG. 8 is a block diagram of an example electronics module of the inhalation device.

FIG. 9A is a perspective view of an example switch contact of the inhalation device.

FIG. 9B is a perspective view of another example switch contact of the inhalation device.

FIG. 10A is a perspective view of an example electronics module of an inhalation device.

FIG. 10B is a perspective view of example contact pads of the example electronics module of FIG. 10A.

FIG. 11A is a perspective view of an example electronics module of an inhalation device.

FIG. 11B is a perspective view of example contact pads of the example electronics module of FIG. 11A.

FIG. 12A is a partial cross-sectional view of the inhalation device of FIG. 1 with the mouthpiece cover in a closed position.

FIG. 12B is a partial cross-sectional view of the inhalation device of FIG. 1 with the mouthpiece cover in an open position to expose the mouthpiece.

FIG. 12C is a partial cross-sectional view of the inhalation device of FIG. 1 with the mouthpiece cover in the open position and in response to a user's inhalation through the mouthpiece.

FIG. 13A-D are example scatterplots of pressure readings with a sensor system located on the front or back of the top or bottom surface of a first portion of a PCB (e.g., of the inhalation device of FIG. 1).

FIG. 14 is a graph of an exemplary relationship between pressure measurements and airflow rates through a flow pathway of an inhalation device.

FIG. 15 is a diagram of an example system including an inhalation device.

### DETAILED DESCRIPTION

The present disclosure describes devices, systems and methods for sensing, tracking and/or processing usage conditions and parameters associated with a drug delivery device. The devices, systems and methods are described in the context of a breath-actuated inhalation device for delivering medication into a user's lungs. However, the described solutions are equally applicable to other drug delivery devices, such as an injector, a metered-dose inhaler, a nebulizer, a transdermal patch, or an implantable.

Asthma and COPD are chronic inflammatory disease of the airways. They are both characterized by variable and recurring symptoms of airflow obstruction and bronchospasm. The symptoms include episodes of wheezing, coughing, chest tightness and shortness of breath. The symptoms are managed by avoiding triggers and by the use of medicaments, particularly inhaled medicaments. The medicaments include inhaled corticosteroids (ICSs) and bronchodilators.

Inhaled corticosteroids (ICSs) are steroid hormones used in the long-term control of respiratory disorders. They function by reducing the airway inflammation. Examples include budesonide, beclomethasone (dipropionate/dipropionate HFA), fluticasone (propionate), mometasone (furoate), ciclesonide and dexamethasone (sodium). Parentheses indicate examples (e.g., preferred) salt or ester forms.

Different classes of bronchodilators target different receptors in the airways. Two commonly used classes are β2-agonists and anticholinergics. β2-Adrenergic agonists (or “β2-agonists”) act upon the β2-adrenoceptors which induces smooth muscle relaxation, resulting in dilation of the bronchial passages. They tend to be categorised by duration of action. Examples of long-acting β2-agonists (LABAs) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β2-agonists (SABA) are albuterol (sulfate) and terbutaline (sulfate).

Typically, short-acting bronchodilators provide a rapid relief from acute bronchoconstriction (and are often called “rescue” or “reliever” medicines), whereas long-acting bronchodilators help control and prevent longer-term symptoms. However, some rapid-onset long-acting bronchodilators may be used as rescue medicines, such as formoterol (fumarate). Thus, a rescue medicine provides relief from acute bronchoconstriction. The rescue medicine is taken as-needed/prn (pro re nata). The rescue medicine may also be in the form of a combination product, e.g. ICS-formoterol (fumarate), typically budesonide-formoterol (fumarate) or beclomethasone (dipropionate)-formoterol (fumarate). Thus, the rescue medicine is preferably a SABA or a rapid-acting LABA, more preferably albuterol (sulfate) or formoterol (fumarate), and most preferably albuterol (sulfate).

Anticholinergics (or “antimuscarinics”) block the neurotransmitter acetylcholine by selectively blocking its receptor in nerve cells. On topical application, anticholinergics act predominantly on the M3 muscarinic receptors located in the airways to produce smooth muscle relaxation, thus producing a bronchodilatory effect. Examples of long-acting muscarinic antagonists (LAMAs) include tiotropium (bromide), oxitropium (bromide), aclidinium (bromide), umeclidinium (bromide), ipratropium (bromide), glycopyrronium (bromide), oxybutynin (hydrochloride or hydrobromide), tolterodine (tartrate), trospium (chloride), solifenacin (succinate), fesoterodine (fumarate) and darifenacin (hydrobromide).

A number of approaches have been taken in preparing and formulating these medicaments for delivery by inhalation, such as via a dry powder inhaler (DPI), a pressurized metered dose inhaler (pMDI) or a nebulizer.

According to the GINA (Global Initiative for Asthma) Guidelines, a step-wise approach can be taken to the treatment of asthma. At step 1, which represents a mild form of asthma, the patient is given an as-needed SABA, such as albuterol sulfate. The patient may also be given an as-needed low-dose ICS-formoterol, or a low-dose ICS whenever the SABA is taken. At step 2, a regular low-dose ICS is given alongside the SABA, or an as-needed low-dose ICS-formoterol. At step 3, a LABA is added. At step 4, the doses are increased and at step 5, further add-on treatments are included such as an anticholinergic or a low-dose oral corticosteroid. Thus, the respective steps may be regarded as treatment regimens, which regimens are each configured according to the degree of acute severity of the respiratory disease.

COPD is a leading cause of death worldwide. It is a heterogeneous long-term disease comprising chronic bronchitis, emphysema and also involving the small airways. The pathological changes occurring in patients with COPD are predominantly localized to the airways, lung parenchyma and pulmonary vasculature. Phenotypically, these changes reduce the healthy ability of the lungs to absorb and expel gases.

Bronchitis is characterized by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localised hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Symptom severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β2-agonists and corticosteroids. The medicaments are often administered as a single therapy or as combination treatments.

Patients are categorized by the severity of their COPD using categories defined in the GOLD Guidelines (Global Initiative for Chronic Obstructive Lung Disease, Inc.). The categories are labelled A-D and the recommended first choice of treatment varies by category. Patients in group A are recommended a short-acting muscarinic antagonist (SAMA) prn or a short-acting β2-aginist (SABA) prn. Patients in group B are recommended a long-acting muscarinic antagonist (LAMA) or a long-acting β2-aginist (LABA). Patients in group C are recommended an inhaled corticosteroid (ICS)+a LABA, or a LAMA. Patients in group D are recommended an ICS+a LABA and/or a LAMA.

Patients suffering from respiratory diseases like asthma or COPD suffer from periodic exacerbations beyond the baseline day-to-day variations in their condition. An exacerbation is an acute worsening of respiratory symptoms that require additional therapy, i.e. a therapy going beyond their maintenance therapy.

For asthma, the additional therapy for a moderate exacerbation are repeated doses of SABA, oral corticosteroids and/or controlled flow oxygen (the latter of which requires hospitalization). A severe exacerbation adds an anticholinergic (typically ipratropium bromide), nebulized SABA or IV magnesium sulfate.

For COPD, the additional therapy for a moderate exacerbation is repeated doses of SABA, oral corticosteroids and/or antibiotics. A severe exacerbation adds controlled flow oxygen and/or respiratory support (both of which require hospitalization). An exacerbation within the meaning of the present disclosure includes both moderate and severe exacerbations.

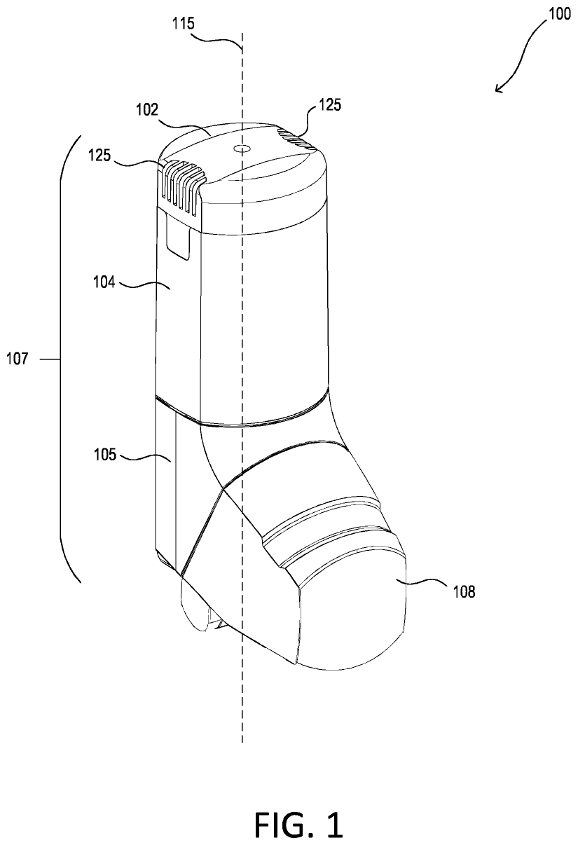

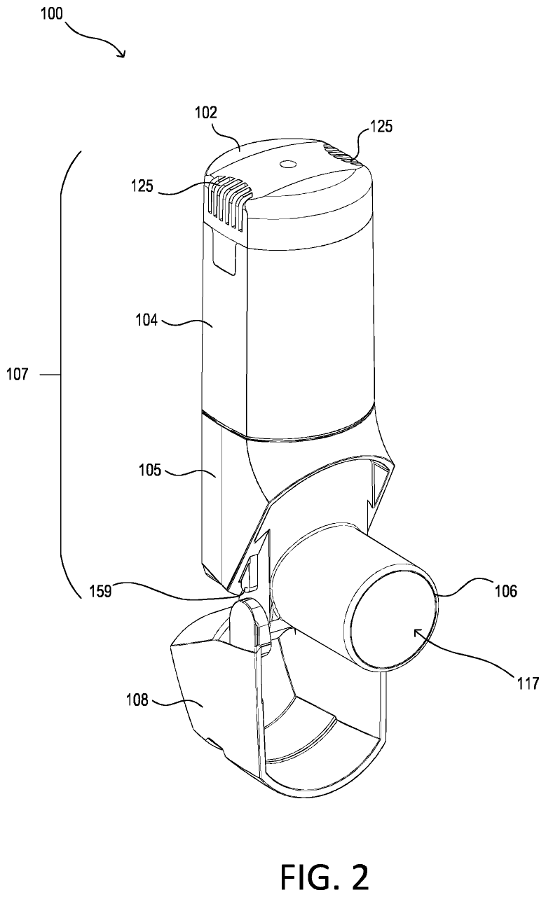

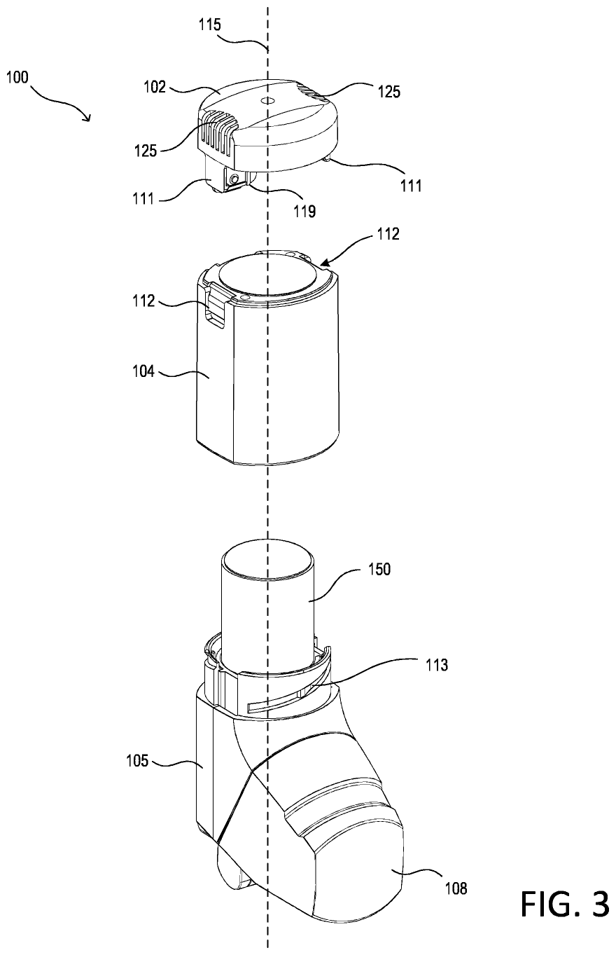

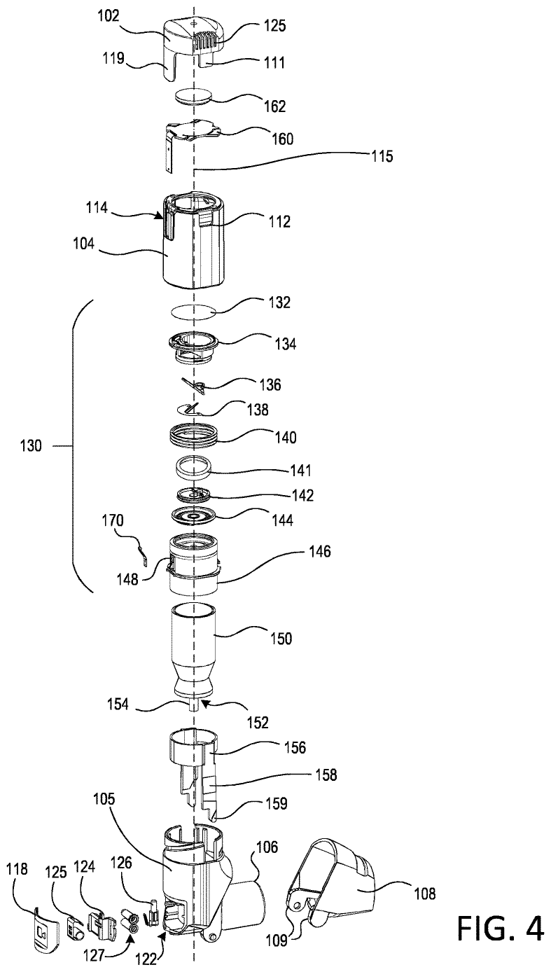

FIG. 1 is a front perspective view of an example inhalation device 100 (e.g., an inhaler). FIG. 2 is a front perspective view of the example inhalation device 100 with a mouthpiece cover 108 in an open position, thereby exposing a mouthpiece 106 of the inhalation device 100. FIG. 3 is a partially exploded view of the inhalation device 100 along a longitudinal axis 115. FIG. 4 is a fully exploded view of the example inhalation device 100 of FIG. 1. The example inhalation device 100 may be a breath-actuated metered-dose inhaler device (e.g., a breath-actuated MIDI). The inhalation device 100 may include a main housing 107. The main housing 107 may include a plurality of portions, such as a top potion 102, a middle portion 104, and a bottom portion 105. Although illustrated as three portions, the main housing 107 of the inhalation device 100 may include less than three portions or more than three portions.

The top portion 102 may be coupled to the middle portion 104 through, for example, a snap-fit connection or a screw-fit connection. In the illustrated example, the top portion 102 may include one or more snaps 111 (e.g., cantilever snaps) and the middle portion 104 may include one or more corresponding snap-in areas 112 that are each configured to securely receive a corresponding snap 111. In some examples, the connection between the top portion 102 and middle portion 104 may be configured such that the connection cannot be broken by a user without undue force. In other examples, the snap-fit connection between the top portion 102 and middle portion 104 may be configured such that the top portion 102 and the middle portion 104 can be separated by a physician or manufacturer, for example, to access one or more electronic components of the inhalation device 100.

The top portion 102 may include a protruding portion 119 that extends from a top surface of the top portion 102 substantially parallel with the longitudinal axis 115. The middle portion 104 may include a corresponding channel 114 that is configured to securely receive the protruding portion 119. The connection between the protruding portion 119 and the channel 114 may be a slide-fit connection (e.g., as illustrated). The top portion 102 may also include a plurality of air vents 125. The air vents 125 may operate as air inlets that allow for air to flow into and through the inhalation device 100, and out of an air outlet 117 in the mouthpiece 106 of the inhalation device 100. Accordingly, the top portion 102 may define an air inlet, the middle portion 104 and the bottom portion 105 may define a hollow interior, and the mouthpiece 106 may define the air outlet 117. Together the air inlet (e.g., the air vents 125), the hollow interior, and the air outlet 117 may define an airflow path from which air may enter and exit the inhalation device 100 to assist with dispensing of the medicament from the mouthpiece 106. Examples of air inlets (e.g., the air vents 125) that may be included in the inhalation device 100 are described in WO 01/93933 A2 and U.S. Pat. No. 8,931,476 B2, the entire disclosures of which are incorporated herein by reference.

The middle portion 104 may define a substantially hollow interior. As described in more detail below, the middle portion 104 may surround (e.g., cover) a movable inner housing 130 of the inhalation device 100. The middle portion 104 may be coupled to the bottom portion 105 through, for example, a twist connection. In the illustrated example, the bottom portion 105 may include a channel 113 that is configured to receive a corresponding protrusion (not shown) on the interior of the middle portion 104 when the middle portion 104 is rotated around the bottom portion 105 to secure the middle and bottom portions 104, 105 together. In some examples, one or both of the middle and bottom portions 104, 105 may comprise longitudinal ribs that extend along the interior of the middle and bottom portions 104, 105 along the longitudinal axis 115.

The bottom portion 105 may include a dose counter system, the mouthpiece 106, the mouthpiece cover 108, and a stem block (not shown) that is configured to receive a valve stem 154 of the medication canister 150. When installed within the main housing 107, the medication canister 150 may elongate along a longitudinal axis 115 within the main housing 107 (e.g., within the middle portion 104 and the bottom portion 105 of the main housing 107). For example, the bottom portion 105 of the main housing 107 may be configured to receive (e.g., securely receive) the medication canister 150. The medication canister 150 may include a metering valve 152 and a valve stem 154. The arrangement of openings in the metering valve 152 of the medication canister 150 may be the same as those described in US 2016/0084385, which is incorporated by reference herein. Further, although illustrated as a breath-actuated MDI that includes medicament in the medication canister 150, in other examples, the inhalation device 100 may include dry powder medicament that is stored in a blister strip or a hopper, such as described in US 2019/0328984 A1 and/or U.S. Pat. No. 11,000,653 B2, the entire disclosures of which are incorporated herein by reference.

As noted herein, the bottom portion 105 may include a stem block (not shown) that includes a nozzle. In some examples, the stem block may be formed as part of the inner surface of the bottom portion 105 of the main housing 107. The valve stem 154 of the metering valve 152 of the medication canister 150 may be configured to be inserted into the stem block. In response to a compressive force on the medication canister 150 (e.g., via a breath-actuated pressure differential (e.g., via a compression spring 140) or physical force by a user), the medication canister 150 may be configured to move axially along the longitudinal axis 115 with respect to the valve stem 154. This degree of axial movement may be sufficient to actuate the metering valve 154 and cause a metered quantity of the medicament (e.g., a dose) to be expelled through the valve stem 154, which is then released into the mouthpiece 106 via the nozzle in the stem block. A user inhaling through the mouthpiece 106 will thus then receive a dose of the medicament. Examples of the stem block and the nozzle are illustrated in U.S. Pat. No. 8,132,712 B2, the entire disclosure of which is incorporated herein by reference.

The dose counter may include a dose counter chamber 122 that includes a dose counter system 124, and which is covered by a dose counter cover 118. The dose counter system 124 may include counter tape 125, an actuation pin 126 and a return spring (not shown). The counter tape 125 (e.g., a ribbon) may rotate around one or more shafts of a chassis preassembly 127 such that a number (e.g., a dose count) that is printed or written on the counter tape 125 may be presented through a window of the dose counter cover 118. The actuation pin 126 may be arranged to engage and be depressed by the medicament canister 150 when the medicament canister 150 is moved to a dose delivery position. As such, the dose counter 124 may be configured to decrement each time the medicament canister 150 is actuated (e.g., by way of a user's inhalation through the mouthpiece 106). The dose counter of the inhalation device 100 may take various forms and may, for example, be as described in EP 2 135 199 A and/or EP 2 514 464 A, the entire disclosures of which are incorporated herein by reference.

The mouthpiece cover 108 may be hinged to the bottom portion 105 of the main housing 107 so that it may open and close to expose the mouthpiece 106. The inhalation device 100 may include a yoke 156 that may, for example, reside with the bottom portion 105 and the middle portion 104 of the main housing 107. The yoke 156 may include drive rods or legs 158 having distal ends 159 which are driven by respective cams 109 on the pivotally-connected mouthpiece cover 108. An example of the yokes is illustrated and described in U.S. Pat. No. 10,792,447 B2, the entire disclosure of which is incorporated herein by reference. Accordingly, the mouthpiece cover 108 may be configured to move between a closed position (e.g., as illustrated in FIG. 1) and an open position (e.g., as illustrated in FIG. 2). As described in more detail here, movement of the mouthpiece cover 108 from the closed position to the open position may prime the inhalation device 100 such that the inhalation device 100 is placed into a prepared configuration where the medication canister 150 is actuatable by patient inhalation induced airflow. Although illustrated as a hinged connection, the mouthpiece cover 108 may be connected to the inhalation device 100 through other types of connections. Further, in some alternate embodiments, the mouthpiece cover 108 may be omitted.

The inhalation device 100 may also include the movable inner housing 130. When assembled, the movable inner housing 130 may reside within an internal cavity of the middle portion 104 of the main housing 107. The movable inner housing 130 may be configured to move with the medication canister 150 when the medication canister 150 is moved to administer a dose of medicament (e.g., at least a portion of the movable inner housing 130 may move along the longitudinal axis 115). In some examples, the movable inner housing 130 may substantially or completely surround the medication canister 150 (e.g., surround at least the upper half of the medication canister 150). In other examples, the movable inner housing 130 may reside adjacent to the medication canister, but not surround the medication canister 150. Further, in some instances, the movable inner housing 130 may be affixed to or integral with the medication canister 150.

The movable inner housing 130 may be a force holding unit that is configured to maintain a force for actuating the medication canister 150 until a user inhales through the mouthpiece 106 of the inhalation device 100. For example, the movable inner housing 130 may be the force holding unit described in U.S. Pat. No. 10,792,447 B2 and/or EP 1 289 589 A, the entire disclosures of which are incorporated herein by reference. For instance, in some examples the movable inner housing 130 may operate substantially similar to the force holding unit disclosed with reference to FIGS. 1 to 3 of EP 1 289 589 A and the yoke 156 and mouthpiece cover 108 may operate substantially similar to that described in EP 2 514 465 A, including but not limited to FIG. 22 thereof, all of which is incorporated herein by reference.

The movable inner housing 130 may be configured to move in response to a dose of medication being dispensed from the medication canister 150. For example, the movable inner housing 130 may be configured to move in response to a user's inhalation through the mouthpiece 106 (e.g., in the instances where the inhalation device 100 is a breath-actuated MDI) or a user action performed on the inhalation device 100, such as the manual compression of the medication canister 150 (e.g., in the instances where the inhalation device 100 is a press-and-breathe MIDI). In some examples, the movable inner housing 130 may be configured to be in a first configuration (e.g., a first position) when the medication canister 150 is in a first position, a second configuration (e.g., a second position) spaced from the first configuration along the longitudinal axis 115 when the medication canister 150 is in a second position, and a third configuration (e.g., a third position) spaced from the first and second configurations along the longitudinal axis 115 when the medication canister 105 is in a third position. The medication canister 150 may be configured to administer a dose of medicament when moved from the second position to the third position. The medication canister 150 may be configured to move from the first position to the second position in response to movement of the mouthpiece cover 108 of the inhalation device 100 from a closed position to an open position to expose the mouthpiece 106 of the inhalation device 100.

The first configuration of the movable inner housing 130 may be a rest configuration in which the metering valve 152 of the medication canister 150 is in a refill position (e.g., a fully extended position). In some examples, the medication canister 150 cannot be actuated to administer a dose of medicament when the movable inner housing 130 in the first configuration. The second configuration of the movable inner housing 130 may be a prepared configuration in which the medication canister 150 is actuatable by patient inhalation induced airflow. The third configuration of the movable inner housing 130 may be an actuation configuration in which the metering valve 152 of the medication canister 150 is in a dose delivery position (e.g., a depressed position such that the medication canister 150 administers, or expels, a dose of medicament).

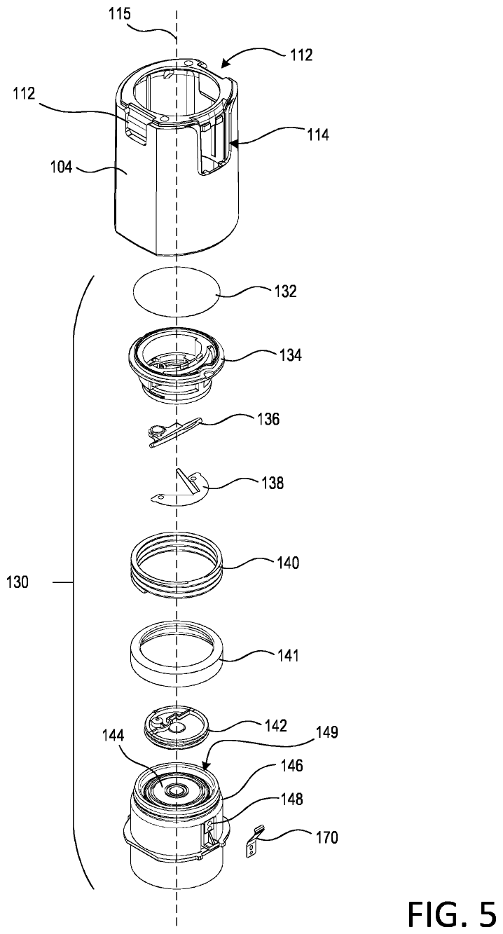

FIG. 5 is a partially exploded view of a movable inner housing 130 (e.g., a force holding unit) of the example inhalation device 100. As noted above, the movable inner housing 130 may reside within an internal cavity of the middle portion 104 of the main housing 107 of the inhalation device 100. In some examples, the movable inner housing 130 include similar components and operate in the same manner as the force holding unit described in U.S. Pat. No. 10,792,447 B2 and/or EP 1 289 589 A, the entire disclosures of which are incorporated herein by reference.

The movable inner housing 130 may include a lower cap 146, an insert 144, a diaphragm 142 attached to an upper surface 149 of the lower cap 146, a retaining ring 141 for the lower cap 146, a compression spring 140, a flap valve spring 138, a pivotally mounted flap valve 136, a flap valve housing 134, and a filter 132. The lower cap 146 may engage the medication canister 150. The pivotally mounted flap valve 136 may be configured to selectively seal a valve port located in the diaphragm 142. As described in more detail herein, an inhalation by a user may cause air to pivot the flap valve 136, which may open a valve channel in the diaphragm 142 allowing passage of the air into a volume between the diaphragm 142 and the lower cap 146 (e.g., thereby causing the volume to reach atmospheric pressure). The passage of air into the volume between the diaphragm 142 and the lower cap 146 may produce a downward motion of the medication canister 150, action (e.g., movement or expansion) of the compression spring 140, and actuation of the metering valve 152 of the medication canister 150 to release a dose through into the mouthpiece 106.

When the inhalation device 100 is in the rest configuration and the mouthpiece cover 108 is in the closed position, the lower cap 146 may be in a first position. When the mouthpiece cover 108 is moved from the closed position to the open position to expose the mouthpiece 106, the lower cap 146 may be forced downwards under the action of the compression spring 140. Then, as the lower cap 146 moves down, an enclosed volume between the diaphragm 142 and the lower cap 146 may be increased by an amount (e.g., a linear amount), and while valve port of the diaphragm 142 remains closed, a pressure difference is created in the enclosed volume (e.g., a pressure difference is created between the enclosed volume and the external atmospheric pressure). The offset of the differential between the pressure in the enclosed volume and atmospheric pressure may result in the lower cap 146 resisting action of the compression spring 140. Downward movement of the lower cap 146 may continue until the force is balanced between the force of the compression spring 140 and the opposing forces of the pressure difference and metering valve 152. The geometry of the movable inner housing 130 is arranged such that the balance occurs before the metering valve 152 has been actuated. As such, the movable inner housing 130 may be placed in a second configuration (e.g., a prepared configured) in which the movable inner housing 130 is actuatable by patient inhalation induced airflow. For example, when the movable inner housing 130 is in the second configuration, the metering valve 152 of the medication canister 150 may be in the refill position (e.g., extended position), but a pressure differential may exist such that the medication canister 150 is actuatable by patient inhalation induced airflow via the mouthpiece 106 of the inhalation device 100. Alternatively, in some examples, when the movable inner housing 130 is in the second configuration, the metering valve 152 of the medication canister 150 may be in a pre-actuation position (e.g., where the metering valve is slightly depressed, but not enough to cause a dose of medicament to be metered from the canister), and a pressure differential may exist such that the medication canister 150 is actuatable by patient inhalation induced airflow via the mouthpiece 106 of the inhalation device 100.

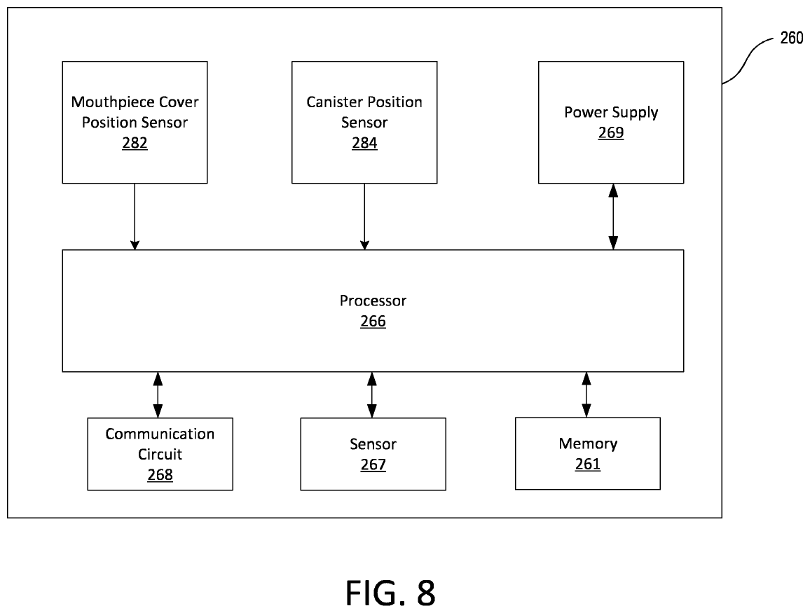

Upon inhalation, air may enter the inhalation device 100 through the air inlets 125 formed on the top portion 102. The flow of air across a vane on the flap valve 136 during inhalation may cause the valve 136 to pivot, which in turn may move a flap valve seal out of its rest position, and open a valve orifice channel in the diaphragm 142. An example of this operation is described with reference to FIG. 8-10 of U.S. Pat. No. 10,792,447 B2, the entire disclosure of which are incorporated herein by reference. The subsequent passage of air into the volume between the diaphragm 142 and the lower cap 146 may allow the volume to reach atmospheric pressure. The resulting imbalance of forces acting on the lower cap 146 and the medication canister 150 may produce the downward (e.g., forward) motion of the medication canister 150 and actuation of the metering valve 152, which in turn may release a measured dose of medicament through the dispensing nozzle and into the mouthpiece 106.

Accordingly, the movable inner housing 130 may rely on the described pressure difference to maintain the second (e.g., prepared) configuration, and release the pressure to move the movable inner housing 130 into the actuation configuration thereby causing the medication canister 150 to release a dose of medicament in response to (e.g., under the action of) the compression spring 140. In such examples, the movable inner housing 130 may be considered pneumatic within the normal meaning of the term. After a dose of medicament is administered by the inhalation device 100, the mouthpiece cover 108 may be returned to the closed position, which in turn may cause the movable inner housing 130 to return to the first configuration and the medication canister 150 to return to the first position. Accordingly, the mouthpiece cover 108 may be opened to prime the medication canister (e.g., prepare the inhalation device 100 for inhalation), and then after inhalation, the mouthpiece cover 108 may be closed to reset the movable inner housing 130 (e.g., return the movable inner housing 130 to a rest configuration). As such, in some examples, at least the lower cap 146, the insert 144, the diaphragm 142, the retaining ring 141, and the compression spring 140 may move (e.g., along the longitudinal axis 115) as the inhalation device 100 transitions between the rest configuration, the prepared configuration, and the actuated configuration. Further, in some instances, the flap valve spring 138 and the pivotally mounted flap valve 136 may move (e.g., in general, but not along the longitudinal axis 115) and the flap valve housing 134 and the filter 132 may not move at all as the inhalation device 100 transitions between the rest, prepared, and actuated configurations.

The inhalation device 100 may also include a switch contact 170, which may be affixed to the movable inner housing 130 (e.g., an outer surface of the movable inner housing 130). For example, the switch contact 170 may be affixed to the lower cap 146, such as to the outer surface of the lower cap 146, for instance, through a snap fit or heat staked coupling. For example, the lower cap 146 may include one or more stakes 148 (e.g., heat stakes) that are configured to be received by one or more openings in the switch contact 170 to secure the switch contact 170 to the lower cap 146. As described in more detail herein, the switch contact 170 may be configured to contact one or more contact pads on an electronic module 160 of the inhalation device 100 in response to, for example, movement of the movable inner housing 130.

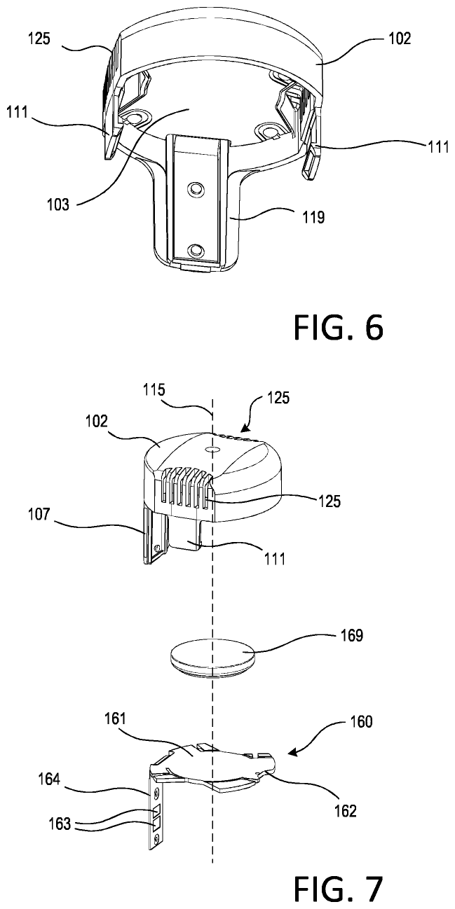

FIG. 6 is a perspective view of the top portion 102 of the main housing 107 of the inhalation device 100 of FIG. 1 without the electronics module 160 installed. FIG. 7 is a partially exploded view of the top portion 102 and an electronics module 160 of the inhalation device 100. The electronics module 160 may reside substantially within a cavity of the top portion 102. In some examples, the electronics module 160 may be irremovable from the main housing 107 of the inhalation device 100. For example, the electronics module 160 may be heat staked to the top portion 102 of the main housing 107. The electronics module 160 may include a printed circuit board (PCB) 161 that includes a processor, a sensor system, a communication circuit (e.g., a wireless communication circuit), and a power supply, such as a battery. Further, in some examples, the electronics module may also include an indicator (e.g., a light emitting diode (LED), a speaker, or the like).

The PCB 161 may define a first portion 162 and a second portion 164, where the second portion 164 of the PCB 161 may be substantially perpendicular to the first portion 162 of the PCB 161. The second portion 164 of the PCB 161 may extend parallel to the longitudinal axis 115. For example, the second portion 164 of the PCB 161 may extend down a rear side of the main housing 107 (e.g., the top portion 102) of the inhalation device 100, where the location of the mouthpiece 106 may define the front side of the inhalation device 100. In some examples, the second portion 164 of the PCB 161 may be flexible, for example, such that the second portion 164 may be bent to achieve the relative configuration between the first and second portions 162, 164 illustrated in FIG. 7. In such examples, the inhalation device 100 may comprise a ridged layer that is placed on the back of the first portion 162 of the PCB 161.

Alternate examples may use two rigid PCBs, and then connectors to connect the two rigid PCBs. But such examples may complicate the design (e.g., both with size and cost) and create additional failure points for the electronics module.

The PCB 161 may define a top surface that is located in closer proximity to the top, interior surface 103 of the top portion 102 than a bottom surface of the PCB 161. Further, the PCB 161 may define a front side that is in closer proximity to the mouthpiece 106 than a back side of the PCB 161. For example, the back side of the PCB 161 may be located at the junction between the first and second portions 162, 164 of the PCB 161.

The components of the electronics module 160 may be mounted on, and electrically coupled to, the PCB 161 (e.g., the first portion 162 of the PCB 161). For example, the processor, the sensor system, the communication circuit and other circuity may be located on a bottom surface of the first portion 162 of the PCB 161 (e.g., an antenna of the communication circuit may be mounted on the bottom surface of the first portion 162). The bottom surface of the first portion 162 may be defined as the side of the first portion 162 that is in closest proximity to the top side of the medication canister 150, while the top surface of the first portion 162 may be the side that is in closest proximity to a battery 169.

The battery 169 may provide power to the components of the electronics module 160. The battery 169 may be secured to the top surface of the first portion 162 of the PCB 161 between the first portion 162 of the PCB 161 and a top, interior surface 103 of the top portion 102 of the main housing 107 of the inhalation device 100. As such, the first portion 162 of the PCB 161 may reside between a top surface 103 of the medication canister 150 and a top, interior surface 103 of the top portion 102 of the main housing 107 of the inhalation device 100, and the second portion 164 of the PCB 161 may extend along an inner, sidewall of the main housing 107 (e.g., of the middle portion 104 of the main housing 107) parallel with the longitudinal axis 115.

The second portion 164 may include one or more contact pads 163 that may be configured to cause one or more components of the electronics module 160 (e.g., such as the processor and/or the sensor system of the electronics module 160) to change power states and/or cause the processor to record events in response to contact by the switch contact 170 that is attached to the movable inner housing 130. For example, in response to movement of the movable inner housing 130, the switch contact 170 may be configured to move from a first position where the switch contact 170 is not contacting the plurality contact pads (e.g., any of the contact pads) to a second position where the switch contact 170 is contacting at least one of the plurality of contact pads of the electronics module 160 to change a power state of one or more of components of the electronics module 160 (e.g., such as a processor and/or a sensor system of the electronics module 160). As noted above, the moveable inner housing 130 may be configured to move in response to a dose of medication being dispensed from the medication canister 150 (e.g., in response to a user's inhalation through the mouthpiece 106).

In some examples, the electronics module 160 may include a plurality of contact pads 163. Each of the contact pads 163 may comprise a planar electrically conductive strip that is attached to the second portion 164 of the PCB 161 of the electronics module 160. The switch contact 170 may be configured to be out of contact (i.e., not in contact) with any of the contact pads 163 when the moveable inner housing 130 is in the first configuration. However, when the movable inner housing 130 is in the second configuration and/or the third configuration, the switch contact 170 may be configured to contact two or more contact pads 163. As noted above, the second configuration of the movable inner housing 130 may be a prepared configuration in which the medication canister 150 is actuatable by patient inhalation induced airflow. The movable inner housing 130 may be configured to be transitioned from the rest configuration (e.g., the first configuration) to the prepared configuration in response to movement of the mouthpiece cover 108 from a closed position to an open position thereby exposing the mouthpiece 106. In some examples, the processor of the electronics module 160 may be configured to record a mouthpiece cover opening event in response to the switch contact 170 coming into contact with one or more contact pads 163 when the movable inner housing 130 is moved from the rest configuration to the prepared configuration.

The third configuration of the movable inner housing 130 may be an actuation configuration in which the metering valve 152 of the medication canister 150 is in a dose delivery position (e.g., a depressed position), such that the medication canister 150 administers, or expels, a dose of medicament out of the mouthpiece 106. Further, in some examples, the processor of the electronics module 160 may be configured to record a canister actuation event in response to the switch contact 170 coming into contact with one or more contact pads 163 when the movable inner housing 130 is moved from the prepared configuration to the actuation configuration. As such, the contact pads 163 may be stationary while the switch contact 170 moves (e.g., with the movable inner housing 130 and/or the medication canister 150) as the inhalation device 100 transitions from the rest configuration, to the prepared configuration, and then to the actuation configuration.

Accordingly, when the movable inner housing 130 is moved from the rest configuration to the prepared configuration, the inhalation device 100 may be configured to record a mouthpiece cover opening event and record one or more signals (e.g., measurements) from the sensor system. And, when the movable inner housing 130 is moved from the prepared configuration to the actuation configuration, the inhalation device 100 may be configured to record a canister actuation event and record one or more signals (e.g., measurements) from the sensor system. As such, the inhalation device 100 is configured to record a mouthpiece cover opening events, a canister actuation event, and one or more signals (e.g., measurements) from the sensor system during every usage event of the inhalation device 100.

FIG. 8 is a block diagram of an example electronics module 260 of an inhalation device. The electronics module 260 may be an example of the electronics module 160. The electronics module 260 may include a processor 266, a sensor system 267, a communication circuit 268, and a power supply 269, such as a battery (e.g., the battery 169). The processor 266 may access information from, and store data in memory 261 of the electronics module 260. The memory 261 may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory 261 may be internal to the processor 266. The processor 266 may also access data from, and store data in, memory that is not physically located within the electronics module 160, such as on a server or a smartphone.

The processor 266 may include a microcontroller, a programmable logic device (PLD), a microprocessor, an application specific integrated circuit (ASIC), a field-programmable gate array (FPGA), and/or any suitable processing device or control circuit. The memory 261 may include computer-executable instructions that, when executed by the processor 266, cause the processor 266 to implement the processes of the electronics module 260 as described herein. When used herein, the terms controller and processor may be used interchangeably.

The sensor system 267 may include one or more sensors, such as one or more pressure sensors, temperature sensors, humidity sensors, acoustic sensors, optical sensors, orientation sensors, and/or the like. The pressure sensor(s) may include a barometric pressure sensor (e.g., an atmospheric pressure sensor), a differential pressure sensor, an absolute pressure sensor, and/or the like. The sensors may employ microelectromechanical systems (MEMS) and/or nanoelectromechanical systems (NEMS) technology. The pressure sensor(s) may be configured to provide an instantaneous pressure reading to the processor 266 of the electronics module 260 and/or aggregated pressure readings over time. The pressure sensor(s) may be configured to measure a plurality of atmospheric pressures within the inhalation device 100. Examples of the sensors 267 are described in reference to US 2020/0360630 A1, the entire disclosure of which are incorporated herein by reference. Further, it should be appreciated that the processor 266 of the electronics module 260 may be configured to convert pressure measurements received from the pressure sensor to a flow rate based on Bernoulli's principle (e.g., the Bernoulli/Venturi effect).

The electronics module 260 (e.g., and/or a computer-readable storage medium comprising instructions that resides on an external device) may use measurements from the sensor system 267 to determine one or more dosing events. For example, the electronics module 260 may be configured to categorize a dosing event as a canister actuation event (e.g., a medicine released event). For instance, the electronics module 260 may be configured to detect that the position of the medication canister has changed in response to the switch contact 170 coming into contact with one or more contact pads 163 when the movable inner housing 130 is moved from the prepared configuration to the actuation configuration (e.g., even after a timeout)). The electronics module 260 may be configured to generate an exhalation event when the one or more signals from the sensor indicate an exhalation occurred (e.g., when the flow rate from the sensor indicate a positive pressure change, for instance, when the sensor system comprises a pressure sensor). The electronics module 260 may be configured to generate a possible air vent block event, such as when the flow rate from the sensor exceeds a particular threshold, such as 200 liters per min (L/min).

Further, in some examples, the electronics module 260 may be configured to compare one or more measurements from the sensor system 267 to one or more threshold values to categorize an inhalation event as a no/low inhalation event, a fair inhalation event, a good inhalation event, an excessive inhalation event, and/or an exhalation event. For example, the electronics module may generate a good inhalation event when the measurements from the sensor system 267 indicate a flow rate in a particular range (e.g., greater than 20 L/min, or between 200 L/min and 20 L/min), generate a no inhalation event when the measurements from the sensor system 267 indicate a flow rate that is less than a threshold value (e.g., 20 L/min), and an excessive inhalation event when the measurements from the sensor system 267 indicate a flow rate that is greater than an upper threshold (e.g., greater than 200 L/min). Further, although described primarily in terms of flow rate, the measurements calculated by the sensor system may be used to calculate inhalation volume and/or inhalation duration, and the thresholds may be inhalation volume thresholds and/or inhalation duration thresholds.

The communication circuit 268 may include a transmitter and/or receiver (e.g., a transceiver), as well as additional circuity (e.g., such as a controller and/or memory). The communication circuit 268 may include a wireless communication circuit. For example, the communication circuit 268 may include a Bluetooth chip set (e.g., a Bluetooth Low Energy chip set), a ZigBee chipset, a Thread chipset, etc. As such, the electronics module 260 may be configured to wirelessly provide data (e.g., the parameters determined by the processor 266, such as pressure measurements, temperature, humidity level, orientation, etc., one or more recorded events, etc.) to an external device, including a smartphone. The external device may include software for processing the received information and for providing compliance and adherence feedback and/or any of the notifications described herein to users of the inhalation device 100 via a graphical user interface (GUI).

The power supply 269 may provide power to the components of the electronics module 260. The power supply 269 may be any suitable source for powering the electronics module 260, such as a coin cell battery, for example. The power supply 269 may be rechargeable or non-rechargeable. The power supply 269 may be secured to the electronics module 260 such that the power supply 269 maintains continuous contact with and/or is in electrical connection with the components of a PCB of the electronics module 260. The power supply 269 may have a battery capacity that may affect the life of the power supply 269. As will be further discussed below, the distribution of power from the power supply 269 to the one or more components of the electronics module 260 may be managed to ensure the power supply 269 can power the electronics module 260 over the useful life of the inhalation device 100 and/or the medication contained therein.

The electronics module 260 may have a plurality of power states, each with respective power consumption levels. For example, the electronics module 260 may be configured to operate in a system off state, a sleep state, and/or an active state. Each of the power states may be defined by different power consumption levels. For example, the electronics module 260 may be configured to operate in a system off state, a sleep state, and/or an active state. The electronics module 260 consumes the least amount of power while in the off state (e.g., no power or just enough to run a clock and/or monitor one or more processor pins in electrical communication with the contact pads 163), consumes more power in the sleep state than the off state (e.g., to drive the memory, the communication circuit, and/or a timer or clock), and consumes more power in the active state than in the sleep or off states (e.g., to drive the processor 266, the sensor system 267, the communication circuit 268, potentially in a faster advertising mode than the sleep state, and/or a timer or clock). Examples of the power states of an inhalation device, such as the inhalation device 100, are described in US 2018/0140786 A1, the entire disclosure of which is incorporated herein by reference.

While the electronics module 260 is in the active state, the electronics module 260 may operate in one or more modes, such as a measurement mode, a data storage/data processing mode, an advertising mode, and/or a connected mode. It should be appreciated that the electronics module 260 may operate in multiple modes at one time (e.g., the modes may overlap).

In the measurement mode, the processor 266 of the electronics module 260 may power on the sensor system 267. The processor 266 may cause the sensor system 267 to take pressure measurement readings, temperature readings, humidity readings, orientation readings, etc. for a predetermined time period (e.g., up to 60 seconds) and/or until the mouthpiece cover 108 is closed or no changes in pressure are detected. The processor 266 may turn off one or more components of the electronics module 260 while the sensor system 267 is capturing readings to further conserve power. The sensor system 267 may sample the readings at any suitable rate. For example, the sensor system 267 may have a sample rate of 100 Hz and thus a cycle time of 10 milliseconds. The sensor system 267 may generate a measurement complete interrupt after the measurement cycle is complete. The interrupt may wake the processor 266 or cause it to turn on one or more components of the electronics module 260. For example, after or while the sensor system 267 is sampling one or more pressure measurements, temperature readings, humidity readings, orientation readings, etc., the processor 266 may process and/or store the data and, if measurements are complete, power off the sensor system 267.

In the data storage/data processing mode, the processor 266 may power on at least a portion of the memory 261 within the electronics module 260. The processor 266 may process the readings from the sensor system 267 to compute, estimate, calculate or otherwise determine parameters (e.g., usage and/or storage conditions) and store the parameters in memory 261. The processor 266 may also compare the readings and/or parameters to one or more thresholds or ranges to assess how the inhalation device 100 is being used and/or the conditions under which the inhalation device 100 is being used. Depending on the results of the comparison, the processor 266 may drive one or more indicators to provide feedback to the user of the inhalation device 100. As noted above, the electronics module 260 may operate in the measurement mode and the data storage/data processing mode simultaneously. After determining one or more parameters (e.g., usage and/or storage conditions) from the readings of the sensor system 267, the processor 266 may transmit the parameters and/or associated timestamps (e.g., based on the internal counter) to the external device when in the connected mode.

In the connected mode, the communication circuit 268 may be powered on and the electronics module 260 may be “paired” with an external device, such as a smartphone. The processor 266 may retrieve data from the memory 261 and wirelessly transmit the data to the external device. The processor 266 may retrieve and transmit all of the data currently stored in the memory 261. The processor 266 may also retrieve and transmit a portion of the data currently stored in the memory 261. For example, the processor 266 may be able to determine which portions have already been transmitted to the external device and then transmit the portion(s) that have not been previously transmitted. Alternatively, the external device may request specific data from the processor 266, such as any data that has been collected by the electronics module 260 after a particular time or after the last transmission to the external device. The processor 266 may retrieve the specific data, if any, from the memory 261 and transmit the specific data to the external device.

Further, when connected with the external device, the electronics module 260 may be configured to transmit Bluetooth special interest group (SIG) characteristics for managing access to data stored in the module 260. The Bluetooth SIG characteristics may include one or more of a manufacturer name of the inhalation device 100, a serial number of the inhalation device 100, a hardware revision number of the inhalation device 100, and/or a software revision number of the inhalation device 100. When connected with the external device, the electronics module 260 may retrieve data from memory 261 and transmit the data to the external device.

The electronics module 160 may include a mouthpiece cover position sensor 282 and a canister position sensor 284. The mouthpiece cover position sensor 282 may be configured to sense the position of the mouthpiece cover 108 (e.g., open or closed), and the canister position sensor 284 may be configured to sense when the position of the medication canister 150 changes (e.g., along the longitudinal axis 115 within the main housing 107, such as in the first, second, or third position). In some examples, the mouthpiece cover position sensor 282 may be a contact pad (e.g., one of the contact pads 163), and the canister position sensor 284 may include one or more contact pads (e.g., one or more of the contact pads 163). As such, the processor 266 may be configured to determine the position of the mouthpiece cover 108 and/or determine when the position of the mouthpiece cover 108 changes based on feedback from the mouthpiece cover position sensor 282. Further, the processor 266 may be configured to determine the position of the medication canister 150 and/or determine when the position of the medication canister 150 changes based on feedback from the canister position sensor 284. The processor 266 may be configured to timestamp and/or transmit data indicating the position of the mouthpiece cover 108 and/or the medication canister 150 to the external device.

The signals generated by the switch contact 170 contacting the contact pads 163 and/or the measurement readings taken by the sensory system 267 may be timestamped and stored in memory 261 of the electronics module 260. The foregoing parameters may be indicative of various usage and/or storage conditions associated with the inhalation device 100. For example, as movement of the movable inner housing 130 causes the switch contact 170 to contact one or more of the contact pads 163, the processor 266 may use the signals from the contact pads 163 to record and timestamp each transition. Further, since the signals from the contact pads 163 may correlate to the position of the mouthpiece cover 108 (e.g., open or closed), the processor 266 may be able to detect and track the position of the mouthpiece cover 108 and/or medicament canister 150 over time. It will be appreciated that the processor 266 may be able to sense and track the status of the mouthpiece cover 108 without interfering with the delivery of medication through the flow pathway of the inhalation device 100.

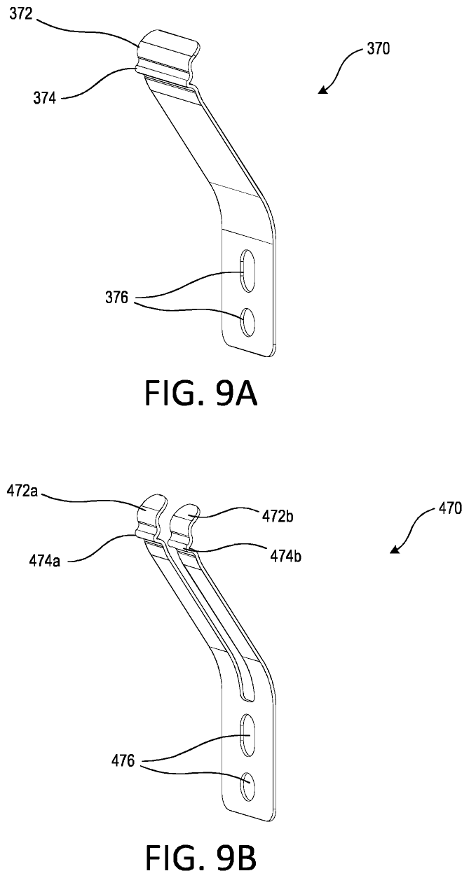

In some switch designs, the switch contact 170 and the switch pads 163 may fall out of contact intermittently, which may cause issues with the electronics module 160, such as one or more components of the electronics module 160 inadvertently changing power states, the inadvertent recording of one or more events, etc. The intermittent electrical contact can be due to a handful of reasons, such as twisting or movement that occurs within the inhalation device 100 when the inhalation device is moved between configurations (e.g., between the rest, prepared, and actuated configurations). In some examples, the compression spring 140 may cause the modified lower cap 146 to twist (e.g., due to the inconsistent downward force caused by the compression spring 140, due to its twisted design), which can cause the switch contact 170 to be off-level. For example, the compression spring 140 may exhibit a slightly different force profile across the bottom of the compression spring 140 based on the rotation of the compression spring 140 when it is installed/inserted into the inhalation device 100. Further, in some examples, the PCB 161 (e.g., the second portion 164 of the PCB 161) of the electronics module 160 may include a plastic component, and the plastic component may not be perfectly flat (e.g., may include dips at the middle or edge), which may cause void areas where the switch contact 170 could lose contact with the contact pads 163. Accordingly, two different switch designs as presented in FIG. 9A and FIG. 9B, and two different switch pad designs are also presented in 10B and 11B, which help solve some of the aforementioned technical problems.

FIG. 9A is a perspective view of an example switch contact 370 of the inhalation device 100. The switch contact 370 may be an example of the switch contact 170 of the inhalation device 100. The switch contact 370 may define a single prong 372. As such, the switch contact 370 may comprise a single, monolithic body. The prong 372 may define a ridge (e.g., nub) 374 that is configured to contact one or more contact pads of an electronics module of the inhalation device (e.g., such as the contact pads 163 of the electronics module 160). The switch contact 370 can be comprised of a material, such as a metal, that is both electrically conductive and capable of flexing/bending such that the switch contact 370 can be flexibly biased against the contact pads of the electronics module to maintain contact therewith. The switch contact 370 which may be affixed to a moveable inner housing of an inhalation device (e.g., such as the movable inner housing 130). For example, the switch contact 370 may be affixed to a lower cap (e.g., the lower cap 146), for instance, through a snap fit or heat staked coupling. For example, the lower cap may include one or more stakes (e.g., the stakes 148) that are configured to be received by one or more openings 376 in the switch contact 370 to secure the switch contact 370 to the lower cap.

As described in more detail below, the prong 372 (e.g., the ridge 374) may be configured to contact multiple contact pads simultaneously when, for example, the movable inner housing is in a second and/or third configuration (e.g., the prepared and/or actuation configuration). For instance, the prong 372 may be configured to be in contact with a first contact pad and a second contact pad when a movable inner housing (e.g., the movable inner housing 130) is in the second configuration and/or when the medication canister is in the second position, and the prong 372 may be configured to be in contact with a third contact pad and the second contact pad when the movable inner housing is in the third configuration and/or when the medication canister is in the third position (e.g., when the metering valve is in a dose delivery position). When the movable inner housing 130 is in the first configuration (e.g., a rest configuration), the prong 372 may be configured to be out of contact (i.e., not in contact) with any of the contact pads of the electronics module.

FIG. 9B is a perspective view of another example switch contact 470 of the inhalation device 100. The switch contact 470 may be an example of the switch contact 170 of the inhalation device 100. The switch contact 470 may define a first prong 472a and a second prong 472b. The first prong 472a may define a first ridge (e.g., nub) 474a that is configured to contact one or more contact pads of an electronics module of the inhalation device (e.g., such as the contact pads 163 of the electronics module 160). The second prong 472b may define a second ridge (e.g., nub) 474b that is configured to contact one or more contact pads of an electronics module of the inhalation device (e.g., such as the contact pads 163 of the electronics module 160).

The switch contact 470 which may be affixed to a moveable inner housing of an inhalation device (e.g., such as the movable inner housing 130). For example, the switch contact 470 may be affixed to a lower cap (e.g., the lower cap 146), for instance, through a snap fit or heat staked coupling. For example, the lower cap may include one or more stakes (e.g., the stakes 148) that are configured to be received by one or more openings 476 in the switch contact 470 to secure the switch contact 470 to the lower cap.

The first prong 472a may be configured to bend (e.g., flex) independently of the second prong 472b, which for example, may enable the first and second prongs 472a, 472b to maintain contact with one or more contact pads of the electronics module. For instance, the first and second prongs 472a, 472b may bend (e.g., flex) independently of one another to compensate for the potential twisting or movement that occurs within the inhalation device when the inhalation device is moved between configurations and/or the imperfections of the internal components of the inhalation device (e.g., of the movable inner housing 130 and/or the second portion 164 of the PCB 161 of the electronics module 160). The first prong 472a may be configured to bend (e.g., flex) independently of the second prong 472b while being affixed to the inhalation device (e.g., the movable inner housing, such as the lower cap) at a single location.

As described in more detail below, the first prong 472a (e.g., the first ridge 474a) may be configured to contact one of a plurality of contact pads while the second prong 472b (e.g., the second ridge 474b) may be configured to contact a different contact pad when, for example, the movable inner housing is in a second and/or third configuration (e.g., the prepared and/or actuation configuration). For instance, the first prong 472a may be in contact with a first contact pad and the second prong 472b may be in contact with a second contact pad when a movable inner housing (e.g., the movable inner housing 130) is in the second configuration and/or when the medication canister is in the second position. Further, the first prong 472a may be in contact with a third contact pad and the second prong 472b may be in contact with the second contact pad when the movable inner housing is in the third configuration and/or when the medication canister is in the third position (e.g., when the metering valve is in a dose delivery position). When the movable inner housing is in the first configuration (e.g., a rest configuration), the first and second prongs 472a, 472b may be configured to be out of contact (i.e., not in contact) with any of the contact pads of the electronics module.

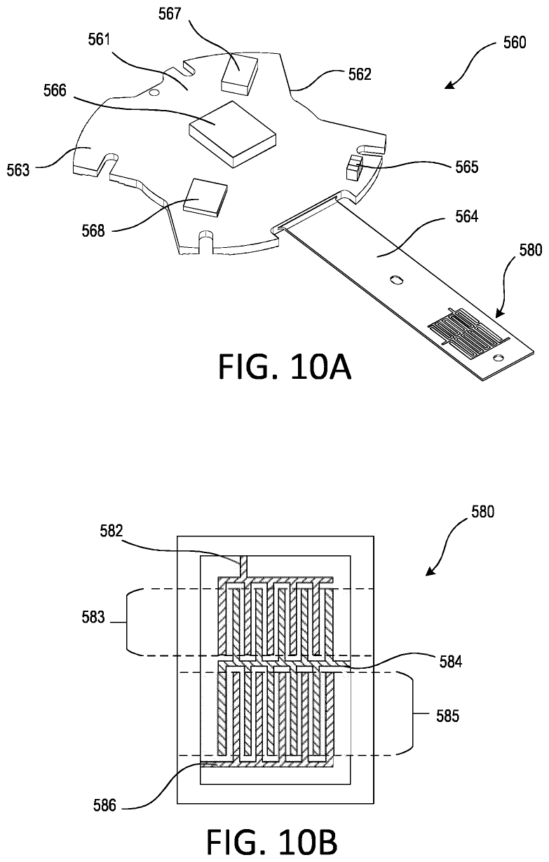

FIG. 10A is a perspective view of an example electronics module 560 of an inhalation device (e.g., the inhalation device 100). FIG. 10B is a perspective view of example contact pads 580 of the example electronics module 560 of FIG. 10A. The electronics module 560 may include a PCB 561 that includes a processor 566, a sensor system 567, a communication circuit 568 (e.g., a wireless communication circuit), and a power supply, such as a battery (e.g., the battery 169). Although not illustrated in FIG. 10A, the processor 566 may be electrically coupled to the sensor system 567, the communication circuit 568, and the power supply. The electronics module 560 may be an example of the electronics module 160 of the inhalation device 100 and/or the electronics module 260. For instance, the processor 566 may be an example of the processor 266, the sensor system 567 may be an example of the sensor 267, and the communication circuit 568 may be an example of the communication circuit 268.

The memory 565 may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory 565 may be internal to the processor 566. The processor 566 may also access data from, and store data in, memory that is not physically located within the electronics module 560, such as on a server or a smartphone. The memory 565 may include computer-executable instructions that, when executed by the processor 566, cause the processor 566 to implement the processes of the electronics module 560 as described herein.

The PCB 561 of the electronics module 560 may define a first portion 562 and a second portion 564. Although illustrated as residing in the same plane with one another, when installed in an inhalation device (e.g., the inhalation device 100), the second portion 564 of the PCB 561 may be substantially perpendicular to the first portion 562 of the PCB 561 (e.g., as shown by the first and second portions 162, 164 of the PCB 161 of the electronics module 160 in FIG. 7). For example, the second portion 564 of the PCB 561 may extend parallel with the longitudinal axis 115 of the inhalation device 100. For example, the second portion 564 of the PCB 561 may extend down a rear side of the main housing 107 (e.g., the top portion 102) of the inhalation device 100, where the location of the mouthpiece 106 may define the front side of the inhalation device 100.

The first portion 562 of the PCB 561 may define a top surface that is located in closer proximity to the top, interior surface of the top portion of the main housing 107 (e.g., the top, interior surface 103 of the top portion 102) than a bottom surface 563 of the first portion 562. Further, the first portion 562 of the PCB 561 may define a front side that is in closer proximity to the mouthpiece 106 that a back side of the first portion 562 of the PCB 561. For example, the back side of the first portion 562 of the PCB 561 may be located at the junction between the first and second portions 562, 564 of the PCB 561.

The components of the electronics module 560 may be mounted on, and electrically coupled to, the first portion 562 of the PCB 561. For example, the processor, the sensor system, the communication circuit and other circuity may be located on the bottom surface 563 of the first portion 562 of the PCB 561. The bottom surface 563 of the first portion 562 may be defined as the side of the first portion 562 that is in closest proximity to the top side of the medication canister, while the top surface of the first portion 562 may be the side that is in closest proximity to the battery. Further, in some examples, the sensor system 567 may be located on the bottom surface 563 of the first portion 562 of the PCB 561 at the front side of the PCB 561 (e.g., as illustrated in FIG. 10A). In other examples, the sensor system 567 may be located on the bottom surface 563 of the first portion 562 of the PCB 561 at the back side of the PCB 561 (e.g., closer to the junction between the first and second portions 562, 564).

As noted above, the inhalation device may include one or more air vents (e.g., the air vents 125 of the top portion 102). Although instructed otherwise, the user may accidentally block one or more of the air vents 125 when they are operating and/or inhaling through the inhalation device, which may cause an increase in the resistance of the inhalation device (e.g., which may cause faulty measurements). Testing has shown that positioning the sensor system 567 on the bottom surface 563 of the first portion 562 of the PCB 561 results in more consistent measurements results (e.g., as compared to positioning the sensor system 567 on the top surface of the first portion 562 of the PCB 561). Further, testing also shows that positioning the sensor system 567 on the front of the bottom surface 563 of the first portion 562 of the PCB 561 results in more consistent measurements results across a variety of different air vent blockage (e.g., as compared to positioning the sensor system 567 on the left, right, or back of the bottom surface 563 of the first portion 562 of the PCB 561). Alternatively, if the front cannot accommodate the sensor system 567, then the sensor system 567 may be located on the back, bottom surface 563 of the first portion 562 of the PCB 561, because the back, bottom surface 563 location showed more consistent measurement results that the left, right, to center of the bottom surface 563 of the first portion 562 of the PCB 561. The processor 566 may be more likely to identify situations where an air vent was accidentally blocked based on the measurement results if the measurement results that occur during an air vent blockage event (e.g., left or right) are more consistent.

The second portion 564 of the PCB 561 may include a plurality of contact pads 580. For example, the contact pads 580 may comprise a first contact pad 582, a second contact pad 584, and a third contact pad 586. Although not illustrated in FIG. 10A, the first, second, and third contact pads 582, 584, 586 may be electrically coupled to the processor 566. Each of the contact pads 582, 584, 586 may comprise a planar electrically conductive strip attached to the second portion 562 of the PCB 561 of the electronics module 560. Further, the second contact pad 584 may be interlaced (e.g., interwoven or intertwined) with the first contact pad 582 (e.g., on a first side) and the third contact pad 586 (e.g., on a second side of the second contact pad 584).

Further, as described in more detail herein, one or more components of the electronics module 560 (e.g., such as the processor 566 and/or the sensor system 567) may be configured to change power states and/or may be configured to record events in response to one or more of the contact pads 580 contacting a switch contact of the inhalation device (e.g., the contact switch 170, the switch contact 270, and/or the switch contact 370). For example, the switch contact may move into a region 583 and contact the first and second contact pads 582, 584 in response to movement of a moveable inner housing (e.g., the movable inner housing 130) from a first configuration to a second configuration, such as in response to a user's actuation or manipulation of the inhalation device (e.g., a movement of the mouthpiece cover from the closed to the open position). When the switch contact is contacting the first and second contact pads 582, 584, an electrical circuit may be closed and one or more components of the electronics module 560 may change power states (e.g., from a sleep or off state to an active state), record one or more events (e.g., a mouthpiece cover opening event), start a timer (e.g., a 60-second timeout timer), and/or begin recording measurements using the sensor system 567.

Similarly, the switch contact may move into a region 585 and contact the second and third contact pads 584, 586 in response to movement of the moveable inner housing from the second configuration to a third configuration (e.g., in response to a user's inhalation that causes an actuation of the medication canister). When the switch contact is contacting the second and third contact pads 584, 586, an electrical circuit may be closed and the electronics module 560 may record one or more events (e.g., a canister actuation event). Further, in some example, when the movable inner housing transitions from the second configuration to the third configuration, the switch contact may remain (e.g., continuously remain) in contact with the second contact pad 584. Accordingly, when the switch contact moves into contact with the second and third contact pads 584, 586, the inhalation device 100 may be configured to record a canister actuation event and record one or more signals (e.g., measurements) from the sensor system.

Finally, the switch contact may be configuration to come out of contact with (e.g., not contact) any of the first, second, or third contact pads 582, 584, 586 when the movable inner housing is moved from the third configuration back to the first configuration (e.g., in response to a movement of the mouthpiece cover from the open position to the closed position). When the switch contact is not in contact with the first, second, or third contact pads 582, 584, 586, the electronics module may change power states (e.g., from the active state back to the sleep state). The contact pads 580 are preferably used with a single prong contact switch, such as the contact switch 370, such that switch contact is configured to contact both the first and second contact pads 582, 584 simultaneously when the switch contact is in the region 583.

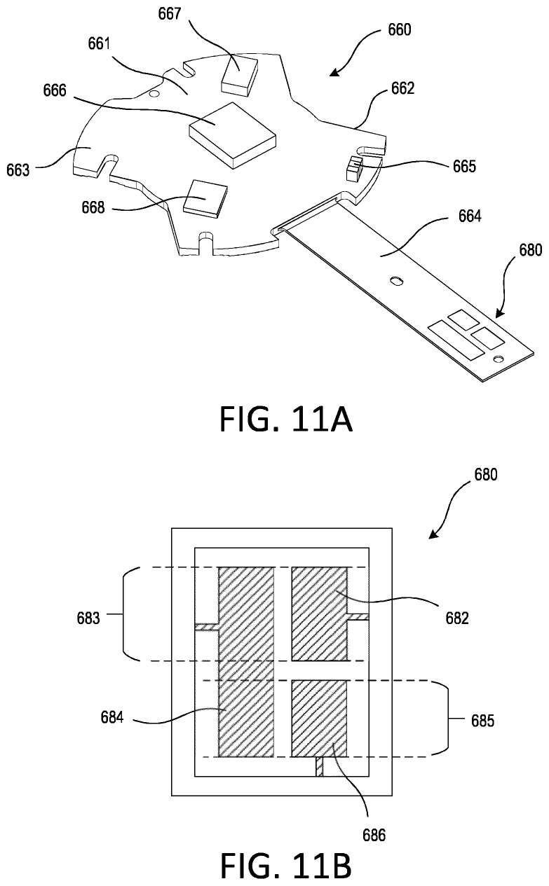

FIG. 11A is a perspective view of an example electronics module 660 of an inhalation device (e.g., the inhalation device 100). FIG. 11B is a perspective view of example contact pads 680 of the example electronics module 660 of FIG. 11A. The electronics module 660 may include a PCB 661 that includes a processor 666, a sensor system 667, a communication circuit 668 (e.g., a wireless communication circuit), and a power supply, such as a battery (e.g., the battery 169). Although not illustrated in FIG. 11A, the processor 666 may be electrically coupled to the sensor system 667, the communication circuit 668, and the power supply. The electronics module 660 may be an example of the electronics module 160 of the inhalation device 100 and/or the electronics module 260. For instance, the processor 666 may be an example of the processor 266, the sensor system 667 may be an example of the sensor 267, and the communication circuit 668 may be an example of the communication circuit 268.

The memory 665 may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory 665 may be internal to the processor 566. The processor 666 may also access data from, and store data in, memory that is not physically located within the electronics module 660, such as on a server or a smartphone. The memory 665 may include computer-executable instructions that, when executed by the processor 666, cause the processor 666 to implement the processes of the electronics module 560 as described herein.

The PCB 661 of the electronics module 660 may define a first portion 662 and a second portion 664. Although illustrated as residing in the same plane with one another, when installed in an inhalation device (e.g., the inhalation device 100), the second portion 664 of the PCB 661 may be substantially perpendicular to the first portion 662 of the PCB 661 (e.g., as shown by the first and second portions 162, 164 of the PCB 161 of the electronics module 160 in FIG. 7). For example, the second portion 664 of the PCB 661 may extend parallel with the longitudinal axis 115 of the inhalation device 100. For example, the second portion 664 of the PCB 661 may extend down a rear side of the main housing 107 (e.g., the top portion 102) of the inhalation device 100, where the location of the mouthpiece 106 may define the front side of the inhalation device 100.

The first portion 662 of the PCB 661 may define a top surface that is located in closer proximity to the top, interior surface of the top portion of the main housing 107 (e.g., the top, interior surface 103 of the top portion 102) than a bottom surface 663 of the first portion 662. Further, the first portion 662 of the PCB 661 may define a front side that is in closer proximity to the mouthpiece 106 that a back side of the first portion 662 of the PCB 661. For example, the back side of the first portion 662 of the PCB 661 may be located at the junction between the first and second portions 662, 664 of the PCB 661.

The components of the electronics module 660 may be mounted on, and electrically coupled to, the first portion 662 of the PCB 661. For example, the processor, the sensor system, the communication circuit and other circuity may be located on the bottom surface 663 of the first portion 662 of the PCB 661. The bottom surface 663 of the first portion 662 may be defined as the side of the first portion 662 that is in closest proximity to the top side of the medication canister, while the top surface of the first portion 662 may be the side that is in closest proximity to the battery. Further, in some examples, the sensor system 667 may be located on the bottom surface 663 of the first portion 662 of the PCB 661 at the front side of the PCB 661 (e.g., as illustrated in FIG. 11A). In other examples, the sensor system 667 may be located on the bottom surface 663 of the first portion 662 of the PCB 661 at the back side of the PCB 661 (e.g., closer to the junction between the first and second portions 662, 664).

As noted above, the inhalation device may include one or more air vents (e.g., the air vents 125 of the top portion 102). Although instructed otherwise, the user may accidentally block one or more of the air vents 125 when they are operating and/or inhaling through the inhalation device, which may cause an increase in the resistance of the inhalation device (e.g., which may cause faulty measurements). Testing has shown that positioning the sensor system 667 on the bottom surface 663 of the first portion 662 of the PCB 661 results in more consistent measurements results (e.g., as compared to positioning the sensor system 667 on the top surface of the first portion 662 of the PCB 661). Further, testing also shows that positioning the sensor system 667 on the front of the bottom surface 663 of the first portion 662 of the PCB 661 results in more consistent measurements results across a variety of different air vent blockage (e.g., as compared to positioning the sensor system 667 on the left, right, or back of the bottom surface 663 of the first portion 662 of the PCB 661). Alternatively, if the front cannot accommodate the sensor system 667, then the sensor system 667 may be located on the back, bottom surface 663 of the first portion 662 of the PCB 661, because the back, bottom surface 663 location showed more consistent measurement results that the left, right, to center of the bottom surface 663 of the first portion 662 of the PCB 661. The processor 666 may be more likely to identify situations where an air vent was accidentally blocked based on the measurement results if the measurement results that occur during an air vent blockage event (e.g., left or right) are more consistent.

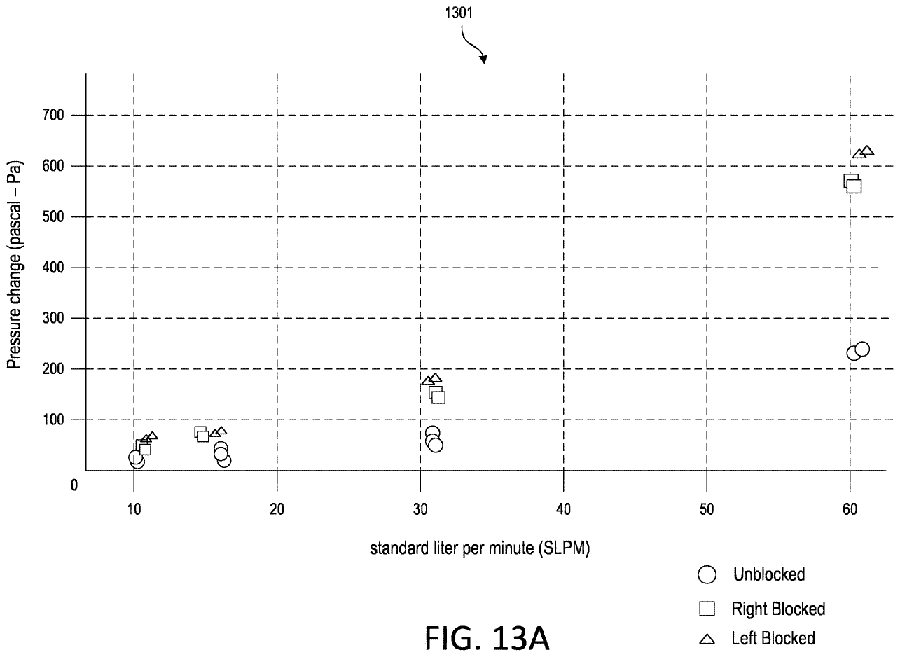

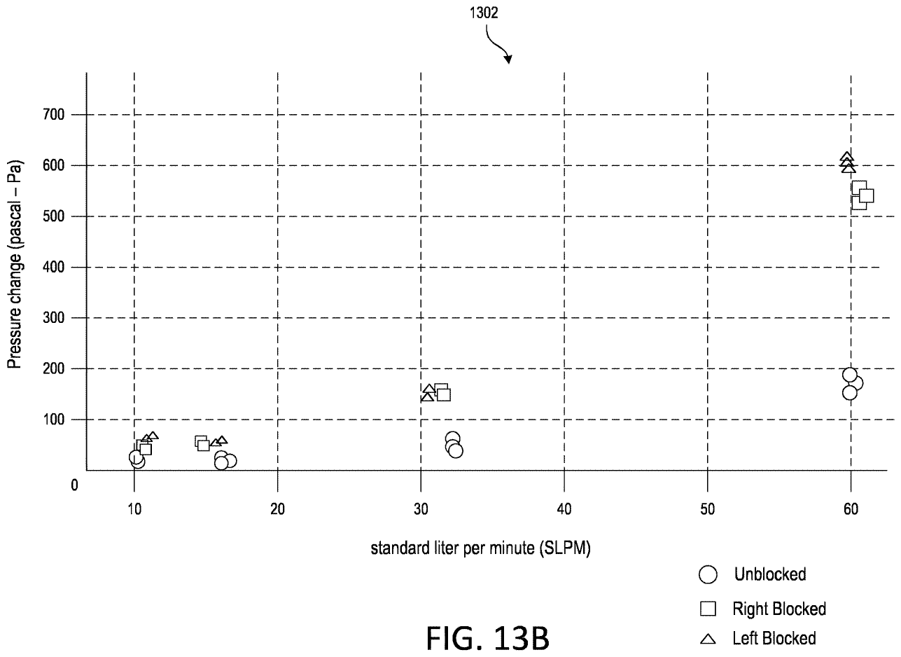

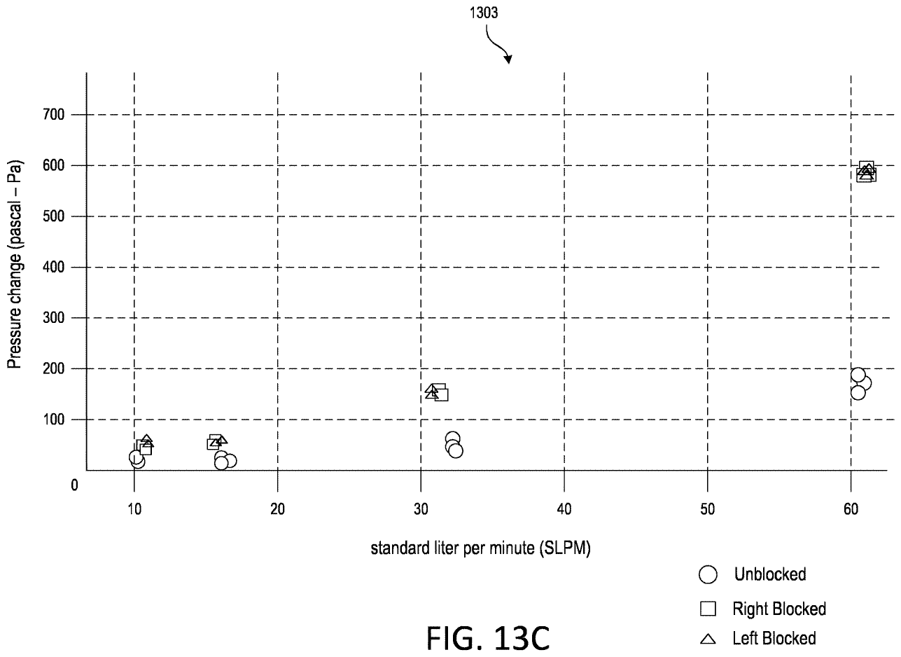

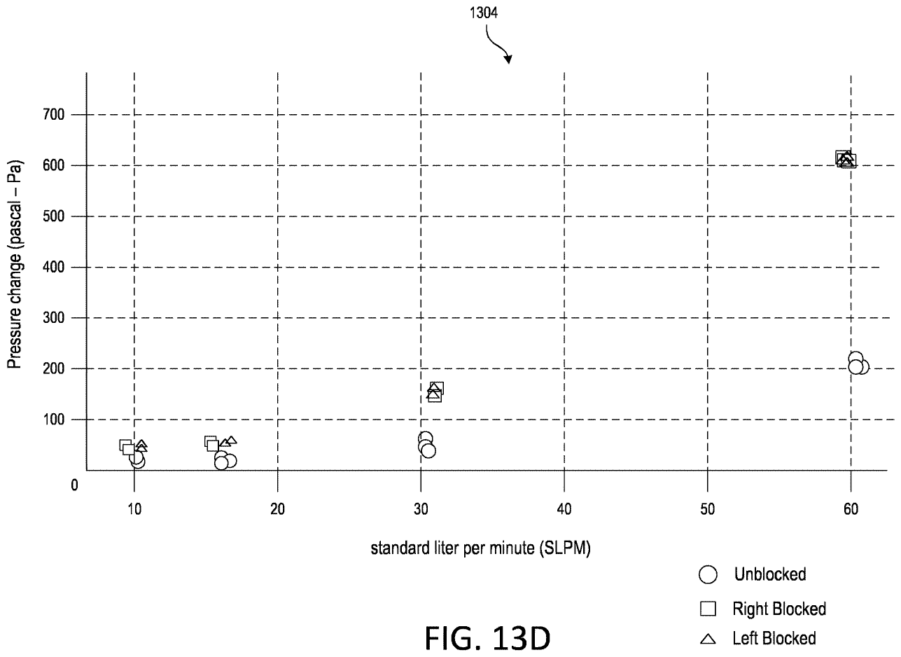

FIG. 13A is an example scatterplot 1301 of pressure readings with a sensor system (e.g., the sensor system 567 or the sensor system 667) located on the back, top surface of the first portion of a PCB (e.g., the first portion 662 of the PCB 661 or the first portion 662 of the PCB 661). The scatterplot 1301 illustrates the pressure readings with the left air vent block (e.g., the air vent 125) illustrated as triangles, pressure readings with the right air vent block (e.g., the air vent 125) illustrated as squares, and pressure readings with neither the left or right vent blocked in circles. FIG. 13B is an example scatterplot 1302 of pressure readings with a sensor system (e.g., the sensor system 567 or the sensor system 667) located on the front, top surface of the first portion of a PCB (e.g., the first portion 662 of the PCB 661 or the first portion 662 of the PCB 661). The scatterplot 1302 illustrates the pressure readings with the left air vent block (e.g., the air vent 125) illustrated as triangles, pressure readings with the right air vent block (e.g., the air vent 125) illustrated as squares, and pressure readings with neither the left or right vent blocked in circles.

FIG. 13C is an example scatterplot 1303 of pressure readings with a sensor system (e.g., the sensor system 567 or the sensor system 667) located on the back, bottom surface of the first portion of a PCB (e.g., the first portion 662 of the PCB 661 or the first portion 662 of the PCB 661). The scatterplot 1303 illustrates the pressure readings with the left air vent block (e.g., the air vent 125) illustrated as triangles, pressure readings with the right air vent block (e.g., the air vent 125) illustrated as squares, and pressure readings with neither the left or right vent blocked in circles. FIG. 13D is an example scatterplot 1304 of pressure readings with a sensor system (e.g., the sensor system 567 or the sensor system 667) located on the front, bottom surface of the first portion of a PCB (e.g., the first portion 662 of the PCB 661 or the first portion 662 of the PCB 661). The scatterplot 1304 illustrates the pressure readings with the left air vent block (e.g., the air vent 125) illustrated as triangles, pressure readings with the right air vent block (e.g., the air vent 125) illustrated as squares, and pressure readings with neither the left or right vent blocked in circles.

A comparison of the scatter plots 1301 and 1302 (e.g., where the sensor system is located on the top of the PCB) to the scatter plots 1303 and 1304 (e.g., where the sensor system is located on the bottom of the PCB) shows that the pressure measurements when the left air vent is blocked are more similar to the pressure measurement when the right air vent is blocked when the sensor system is located on the bottom of the PCB as opposed to the top of the PCB (e.g., and particularly so when the sensor system is located on the front, bottom surface of the PCB, as shown by the measurements in FIG. 13D). As such, positioning the sensor system on the bottom surface of the first portion of the PCB results in more consistent measurements results (e.g., as compared to positioning the sensor system on the top surface of the first portion of the PCB). Further, as illustrated by the example test results illustrated in FIGS. 13A-D, positioning the sensor system on the front of the bottom surface of the first portion of the PCB results in more consistent measurements results across a variety of different air vent blockage (e.g., as compared to positioning the sensor system on the left, right, or back of the bottom surface of the first portion of the PCB).

Referring back to FIGS. 11A-B, the second portion 664 of the PCB 661 may include a plurality of contact pads 680. For example, the contact pads 680 may comprise a first contact pad 682, a second contact pad 684, and a third contact pad 686. Although not illustrated in FIG. 11A, the first, second, and third contact pads 682, 684, 686 may be electrically coupled to the processor 666. Each of the contact pads 682, 684, 686 may comprise a planar electrically conductive strip attached to the second portion 662 of the PCB 661 of the electronics module 660. The first, second, and third contact pads 682, 684, 686 may be substantially rectangularly shaped. In some examples, the first contact pad 682 may be located above the third contact pad 686 on the second portion 662 of the PCB 661, and the first contact pad 682 may be located adjacent the second contact pad 684. Further, the third contact pad 686 may be located below the first contact pad on the second portion 662 of the PCB 661 and adjacent the second contact pad 684. The first, second, and third contact pads 682, 684, 686 may each define a respective longitudinal length (e.g., parallel with the longitudinal axis 115 when the electronics module 660 is installed in the inhalation device 100). In such instances, the longitudinal length of the second contact pad 684 may be substantially equal to a length defined between an upper, transverse edge of the first contact pad 682 and a lower, transverse edge of the third contact pad 686 (e.g., as shown in FIG. 11B). Further, in some examples, the longitudinal length of the second contact pad 684 may be greater than the longitudinal lengths of the first and third contact pads 682, 686 combined.

Further, as described in more detail herein, one or more components of the electronics module 660 (e.g., such as the processor 666 and/or the sensor system 667) may be configured to change power states and/or may be configured to record events in response to one or more of the contact pads 680 contacting a switch contact of the inhalation device (e.g., the contact switch 170, the switch contact 270, and/or the switch contact 370). For example, the switch contact may move into a region 683 and contact the first and second contact pads 682, 684 in response to movement of a moveable inner housing (e.g., the movable inner housing 130) from a first configuration to a second configuration, such as in response to a user's actuation or manipulation of the inhalation device (e.g., a movement of the mouthpiece cover from the closed to the open position). When the switch contact is contacting the first and second contact pads 682, 684, an electrical circuit may be closed and one or more components of the electronics module 660 may change power states (e.g., from a sleep state to an active state), record one or more events (e.g., a mouthpiece cover opening event), start a timer (e.g., a 60-second timeout timer), and/or begin recording measurements using the sensor system 667.

Similarly, the switch contact may move into a region 685 and contact the second and third contact pads 684, 686 in response to movement of the moveable inner housing from the second configuration to a third configuration (e.g., in response to a user's inhalation that causes an actuation of the medication canister). When the switch contact is contacting the second and third contact pads 684, 686, an electrical circuit may be closed and the electronics module 660 may record one or more events (e.g., a canister actuation event). Further, in some example, when the movable inner housing transitions from the second configuration to the third configuration, the switch contact may remain (e.g., continuously remain) in contact with the second contact pad 684.

Finally, the switch contact may be configuration to come out of contact with (e.g., not contact) any of the first, second, or third contact pads 682, 684, 686 when the movable inner housing is moved from the third configuration back to the first configuration (e.g., in response to a movement of the mouthpiece cover from the open position to the closed position). When the switch contact is not in contact with the first, second, or third contact pads 682, 684, 686, the electronics module may change power states (e.g., from the active state back to the sleep state).

The contact pads 680 may be used with a single prong contact switch, such as the contact switch 370, or with a multiple prong contact switch, such as the two-prong contact switch 470. For example, when the two-prong contact switch 470 is used, the first prong 472a (e.g., the first ridge 474a) may be configured to contact the first or third contact pad 682, 686, and the second prong 472b (e.g., the second ridge 474b) may be configured to contact the second contact pad 684. For example, when the switch contact 470 is in a first position (e.g., the movable inner housing is in the first configuration), the first and second prongs 472a, 472b may be configured to not be in contact with any of the first, second, or third contact pads 682, 684, 686. When the switch contact 470 is in the second position (e.g., the movable inner housing is in the second configuration), the first prong 472a may be in contact with the first contact pad 682, and the second prong 472b may be in contact with the second contact pad 684 (e.g., and a metering valve of the medication canister may be in a refill position or a pre-actuation position). And, when the switch contact 470 is in a third position (e.g., the movable inner housing is in the third configuration), the first prong 472a may be in contact with the third contact pad 686, and the second prong 472b may be in contact with the second contact pad 684 (e.g., and the metering valve of the medication canister may be in a dose delivery position).

Further, regardless of whether a single or multi-pronged switch contact is used, when the switch contact transitions from the second position to the third position, the switch contact (e.g., either the single prong or the second prong) may stay in continuous contact with the second contact pad 684.

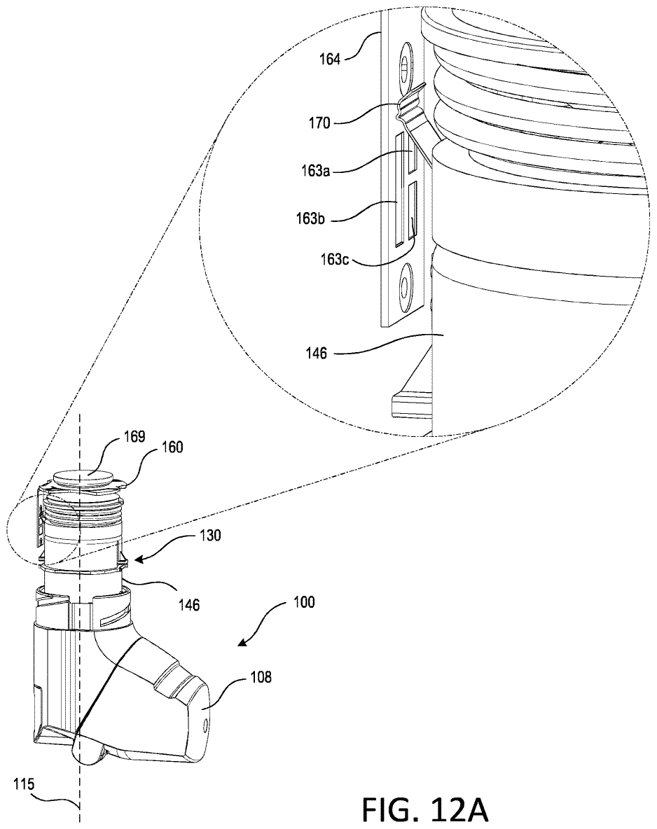

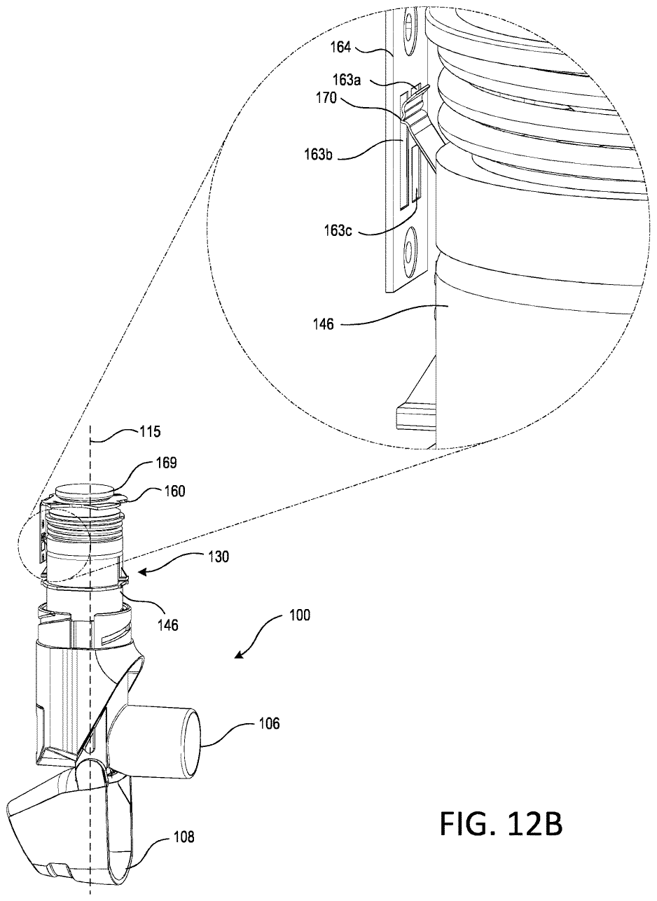

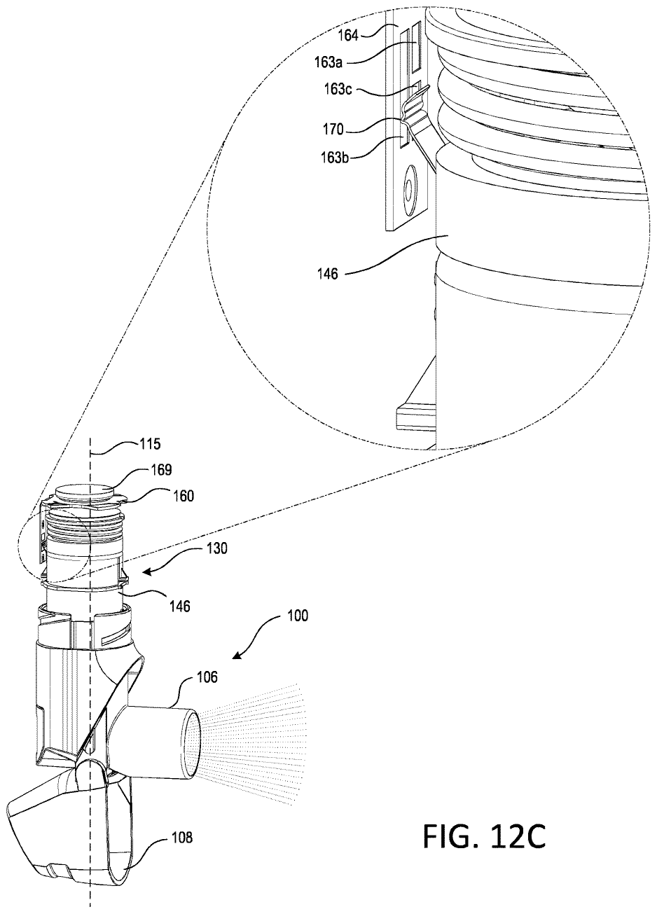

FIG. 12A is a partial cross-sectional view of the inhalation device 100 of FIG. 1 with the mouthpiece cover 108 in a closed position. FIG. 12B is a partial cross-sectional view of the inhalation device 100 of FIG. 1 with the mouthpiece cover 108 in an open position to expose the mouthpiece 106. FIG. 12C is a partial cross-sectional view of the inhalation device 100 of FIG. 1 with the mouthpiece cover 108 in the open position and in response to a user's inhalation through the mouthpiece 106 (e.g., with the metering valve 152 of the medication canister 150 in the dose delivery position). FIGS. 12A-C illustrate the inhalation device 100 without the top or middle portions 102, 104 of the main housing 107 and with the movable inner housing 130 exposed, for example, to help further illustrate and explain the interaction between the switch contact 170 and the contact pads 163 of the electronics module 160 through various states of the inhalation device 100.

FIG. 12A-C illustrate the inhalation device 100 with the switch contact 170 as a single prong switch contact, such as the single prong switch contact 370 of FIG. 9A. However, it should be appreciated that in other examples, the switch contact 170 may be a multi-pronged switch contact, such as the two-pronged switch contact 470 of FIG. 9B. Further, FIG. 12A-C illustrate the electronics module 160 comprising three contact pads, a first contact pad 163a, a second contact pad 163b, and a third contact pad 163c. Although illustrated as three contact pads 163a, 163b, 163c, the electronics module 160 may include more or less than three contact pads. Further, although the contact pads 163a, 163b, 163c are illustrated in a size and configuration similar to that shown in FIG. 11B with the contacts pads 682, 684, 686, in other examples, the contact pads 163a, 163b, 163c may take a size and configuration similar to that shown in FIG. 10B with the contact pages 582, 584, 586.

Referring to FIG. 12A, the inhalation device 100 may be in a rest configuration. For example, the movable inner housing 130 may be in a rest configuration and the metering valve 152 of the medication canister 150 may be in a refill position. When in the refill position, the metering valve 152 may be fully extended and the medication canister 150 may not be expelling (e.g., administering) medication. Accordingly, in FIG. 12A, the movable inner housing 130 may be in a first configuration (e.g., the refill configuration) and the medication canister 150 may be in a first position (e.g., the refill position) along the longitudinal axis 115. The mouthpiece cover 108 may be in the closed position when the inhalation device 100 is in the rest configuration. As such, the rest configuration may be the configuration that the inhalation device 100 is kept when not in use.

Further, as noted above, the switch contact 170 may be coupled to the movable inner housing 130 of the inhalation device 100. When the inhalation device 100 is in the rest configuration, such as in FIG. 12A, the switch contact 170 may be configured to be out of contact (e.g., not in contact) with any of the first, second, or third contact pads 163a, 163b, 163c of the electronics module 160. As such, when the inhalation device 100 is in the rest configuration, the electronics module 160 may be in a sleep or off state. Therefore, the inhalation device 100 may be configured to minimize battery usage when in the rest configuration.

In response to a user action upon the inhalation device, such as a movement of the mouthpiece cover 108 from the closed position (e.g., as shown in FIG. 12A) to the open position (e.g., as shown in FIG. 12B) to expose the mouthpiece 106 of the inhalation device 100, the inhalation device 100 may be placed in a second configuration (e.g., a prepared configuration). For example, in response to the mouthpiece cover 108 being moved from the closed position to the open position, the movable inner housing 130 may be configured to move from the first configuration (e.g., the rest configuration) to a second configuration (e.g., the prepared configuration). Further, in some examples, the movement of the mouthpiece cover 108 from the closed position to the open position may cause the medication canister 150 to move from the first position (e.g., the refill position) to a second position (e.g., a prepared position) along the longitudinal axis of the inhalation device (e.g., the longitudinal axis 115).

When the mouthpiece cover 108 is moved from the closed position to the open position to expose the mouthpiece 106, the lower cap 146 may be forced downwards under the action of the compression spring 140. Then, as the lower cap 146 moves down, an enclosed volume between the diaphragm 142 and the lower cap 146 may be increased by an amount (e.g., a linear amount), and while valve port of the diaphragm 142 remains closed, a pressure difference is created in the enclosed volume (e.g., a pressure difference is created between the enclosed volume and the external atmospheric pressure). The offset of the differential between the pressure in the enclosed volume and atmospheric pressure may result in the lower cap 146 resisting action of the compression spring 140. Downward movement of the lower cap 146 may continue until the force is balanced between the force of the compression spring 140 and the opposing forces of the pressure difference and metering valve 152. The geometry of the movable inner housing 130 is arranged such that the balance occurs before the metering valve 152 has been actuated. As such, the movable inner housing 130 may be placed in the second configuration (e.g., the prepared configured) in which the movable inner housing 130 is actuatable by patient inhalation induced airflow. For example, when the movable inner housing 130 is in the second configuration, the metering valve 152 of the medication canister 150 may be in the refill position (e.g., extended position), but a pressure differential may exist such that the medication canister 150 is actuatable by patient inhalation induced airflow via the mouthpiece 106 of the inhalation device 100. Accordingly, the metering valve 152 of the medication canister 150 may be in the refill position (e.g., extended position) when the medication canister 150 is in the rest position and in the prepared position. Alternatively, in some examples, when the movable inner housing 130 is in the second configuration, the metering valve 152 of the medication canister 150 may be in a pre-actuation position (e.g., where the metering valve is slightly depressed, but not enough to cause a dose of medicament to be metered from the canister).

When the medication canister 150 is in the prepared position, the metering valve 152 may be configured to not expel (e.g., administer) medication. For instance, the metering valve 152 may be in the same position as when the medication canister is in the refill position, or the metering valve 152 may be slightly depressed but not depressed enough to cause medication to be expelled (e.g., administered) from the medication canister 150 (e.g., in the pre-actuation position). However, as noted above, when the medication canister 150 is in the prepared position, the medication canister 150 may be actuatable by patient inhalation induced airflow, for example, since the force is balanced between the force of the compression spring 140 and the opposing forces of the pressure difference and metering valve 152. This occurs before the metering valve 152 has been actuated. As such, the movable inner housing 130 may be placed in a second configuration (e.g., a prepared configured) in which the movable inner housing 130 is actuatable by patient inhalation induced airflow.

Referring still to FIG. 12B, when the inhalation device 100 is in the prepared configuration, the switch contact 170 may be in contact with the first and second contact pads 163a, 163b. For example, the movement of the movable inner housing 130 that occurs in response to the movement of the mouthpiece cover 108 from the closed position to the open position may cause the switch contact 170 to move into contact with the first and second contact pads 163a, 163b. When the switch contact 170 comes into contact with the first and second contact pads 163a, 163b, the electronics module 160 may change power states, such as from a sleep or off state to an active state. When in the active state, the processor may be powered on and/or the sensor system may be powered on and begin recording measurements. Further, in response to the change in power states, the processor of the electronics module 160 may record one or more events, such as a mouthpiece cover opening event.

In response to a user inhalation via the mouthpiece 106 of the inhalation device 100, the inhalation device 100 may be placed in a third configuration (e.g., an actuation configuration), for example, as illustrated in FIG. 12C. For example, in response to the user inhalation, the movable inner housing 130 may be configured to move from the second configuration (e.g., the prepared configuration) to the third configuration (e.g., the actuation configuration). Further, in some examples, the user inhalation may cause the medication canister 150 to move from the second position (e.g., the prepared position) to a third position (e.g., a dose delivery position) along the longitudinal axis of the inhalation device (e.g., the longitudinal axis 115). As such, when the user inhales through the mouthpiece 106, the movable inner housing 130 and the medication canister 150 may move along the longitudinal axis 115 such that the metering valve 152 is moved into the dose delivery position (e.g., a depressed position) and the medication canister 150 administers, or expels, a dose of medicament out of the mouthpiece 106.

For instance, in some examples, a user inhalation may cause air to pivot the flap valve 136, which may open a valve channel in the diaphragm 142 allowing passage of the air into a volume between the diaphragm 142 and the lower cap 146 (e.g., thereby causing the volume to reach atmospheric pressure). The passage of air into the volume between the diaphragm 142 and the lower cap 146 may produce a downward motion of the medication canister 150, action (e.g., movement or expansion) of the compression spring 140, and actuation of the metering valve 152 of the medication canister 150 to release a dose through into the mouthpiece 106. Accordingly, the medication canister 150 may be configured to administer a dose of medicament when moved from the second position to the third position along the longitudinal axis 115, which may occur as a result of a user inhalation through the mouthpiece 106 of the inhalation device 100.

Referring still to FIG. 12C, when the inhalation device 100 is in the actuation configuration, the switch contact 170 may be in contact with the second and third contact pads 163b, 163c. For example, the movement of the movable inner housing 130 that occurs in response to user inhalation may cause the switch contact 170 to move from being in contact with the first and second contact pads 163a, 163b to being in contact with the second and third contact pads 163b, 163c. When the switch contact 170 comes into contact with the second and third contact pads 163b, 163c, the processor of the electronics module 160 record one or more events, such as a canister actuation event. Further, when the user inhales through the mouthpiece 106, the sensor of the electronics module 160 may record the resulting change (e.g., a pressure change in the examples where the sensor system comprises a pressure sensor). Accordingly, the inhalation device 100 is configured to record a mouthpiece cover opening events, a canister actuation event, and one or more signals (e.g., measurements) from the sensor system during every usage event of the inhalation device 100.

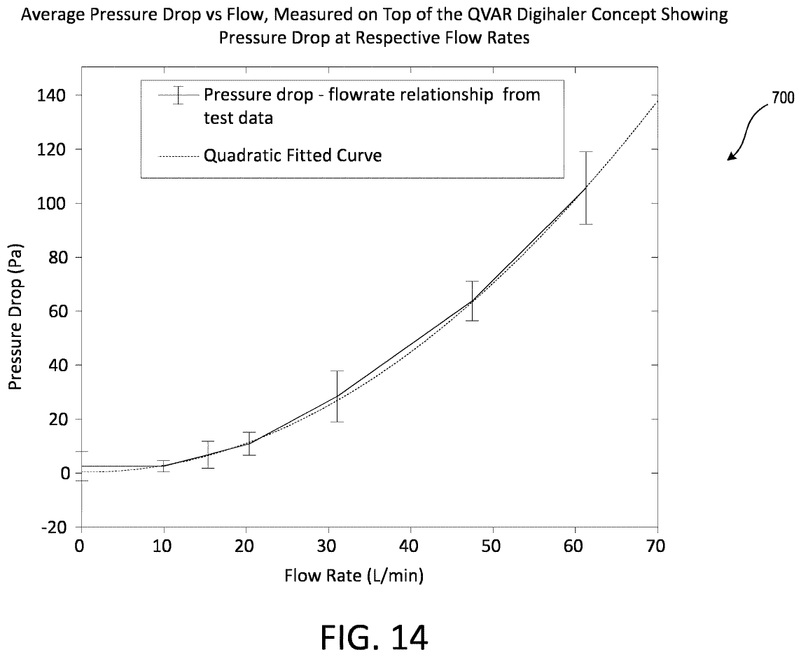

FIG. 14 shows an example of airflow rates based on various pressure measurements calculated by a sensor system of an electronic module of an inhalation device, such as the inhalation device 100. It will be appreciated that the graph 700 of airflow rates and pressure drops shown in FIG. 14 are merely examples, and may vary based on the size, shape, and design of the inhalation device 100 and its internal components.

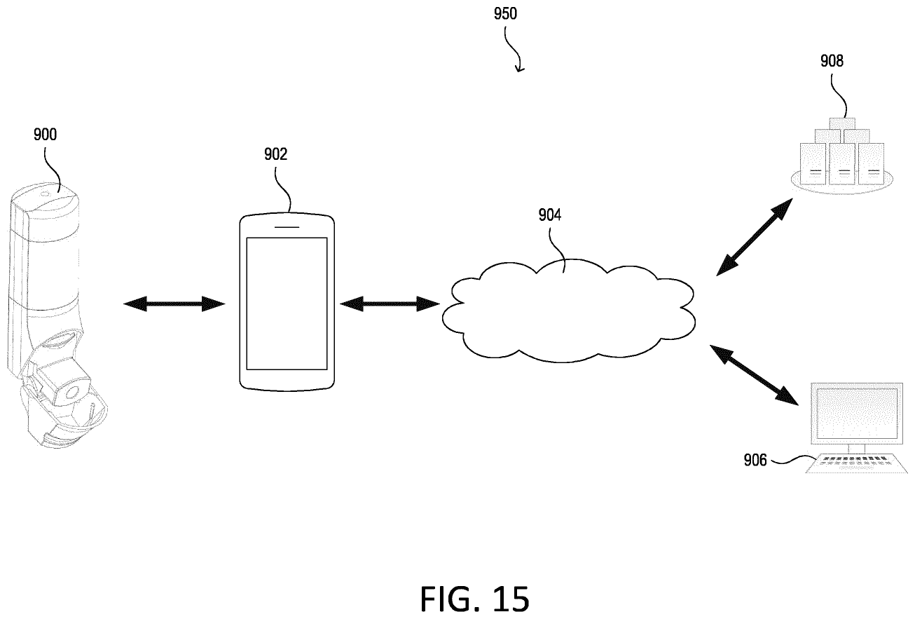

FIG. 15 is a diagram of an example system 950 including the inhalation device 900, an external device 902 (e.g., a mobile device), a public and/or private network 904 (e.g., the Internet, a cloud network), a computer or server 908 associated with a health care provider, such as a hospital or hospital system, a health system, a medical group, a physician, a clinic, and/or a pharmaceutical company. The system 950 also includes a digital health platform (DHP) 906 that resides on one or more servers, and may include computer software configured to perform the functions described in relation to the DHP 906.

The inhalation device 900 may be an example of the inhalation device 100. For example, the inhalation device 900 may include an electronics module (e.g., the electronics module 160, the electronics module 260, the electronics module 560, and/or the electronics module 660). Accordingly, the inhalation device 900 may include a sensor system (e.g., the sensor system 267, the sensor system 567, and/or the sensory system 667). As such, the inhalation device 900 may be configured to record measurements (e.g., pressure measurements, acoustic measurements, etc.) that occur before, during, and/or after an inhalation by a user through the mouthpiece of the inhalation device 900. Further, the inhalation device 900 may be configured to record one or more events, such as a mouthpiece cover opening event, a canister actuation event, inhalation events, and/or one or more error events. The inhalation device 900 may associate a time stamp with the events and/or measurements, and store the events, measurements, and/or timestamps in memory of the electronics module. The inhalation device 900 may include a communication circuit (e.g., the communication circuit 268, the communication circuit 568, and/or the communication circuit 668) that is configured to transmit data, such as the record events, measurements, time stamps, etc. to the external device 902. Further, the inhalation device 900 may receive data from the external device 902, such as, for example, program instructions, operating system changes, dosage information, alerts or notifications, acknowledgments, etc.

The external device 902 may include a smart phone (e.g., an iPhone® smart phone, an Android® smart phone, or a Blackberry® smart phone), a personal computer, a laptop, a wireless-capable media device (e.g., MP3 player, gaming device, television, a media streaming devices (e.g., the Amazon Fire TV, Nexus Player, etc.), etc.), a tablet device (e.g., an iPad® hand-held computing device), a Wi-Fi or wireless-communication-capable television, or any other suitable Internet-Protocol-enabled device. For example, the external device 902 may be configured to transmit and/or receive RF signals via a Wi-Fi communication link, a Wi-MAX communications link, a Bluetooth® or Bluetooth Smart communications link, a near field communication (NFC) link, a cellular communications link, a television white space (TVWS) communication link, or any combination thereof. The external device 902 may transfer data through the public and/or private network 904 to the DHP 906 using, for example, a dedicated API. For example, the external device 902 may send usage data relating to one or more inhalation devices 900 to the DHP 906.

The external device 902 may comprise a computer-readable storage medium that comprises instructions that are associated with the inhalation device 900. The external device 902 (e.g., the computer-readable storage medium comprising the instructions that resides on the external device 902) may be configured to process and/or analyze the data received from the inhalation device 900 to determine the usage parameters associated with the inhalation device 100. For instance, in some examples, the data stored in the memory of the electronics module of the inhalation device 900 (e.g., the signals generated by the switch 170, the measurement readings taken by the sensor system 267, the parameters computed by the processor of the electronics module 160, one or more recorded events, and/or the associated timestamps) may be transmitted to the external device 902, and the external device 902 may process and analyze the data to determine the usage parameters and/or categorize the events associated with the inhalation device 900. Accordingly, in some examples, the external device 902 may receive the measurement data from the inhalation device 900, and the external device 902 may determine one or more events based on the received data, such as no inhalation events, low inhalations events, good inhalation events, excessive inhalation events and/or exhalation events. The external device 902 may also process the data to identify underuse events, overuse events and optimal use events. The external device 902 may further process the data to estimate the number of doses delivered and/or remaining and to identify error conditions, such as those associated with a timestamp error flag. As such, some of the processing that could be performed at the inhalation device 902 may be offloaded to the external device 902.

In some examples, the inhalation device 900 and/or the external device 902 may record (e.g., store) all the data associated with a single usage or inhalation event of the inhalation device 900, for example, based on the timestamps associated with the data (e.g., all timestamps that are within a particular time of one another may be categorized together). For instance, the inhalation device 900 and/or the external device 902 may record an inhalation event that includes the mouthpiece cover opening event, the canister actuation event, any errors events, the one or more signals from the sensor indicative of the user's inhalation, and/or the associated timestamps. The inhalation device 900 and/or the external device 902 may send the inhalation event to a remote server, such as the DHP 906 (e.g., send the mouthpiece cover opening event, the canister actuation event, any errors events, the one or more signals from the sensor indicative of the user's inhalation, and/or the associated timestamps as an inhalation event to the DHP 906).

The inhalation device 900 and/or the external device 902 (e.g., via a computer-readable storage medium comprising executable instructions residing on the external device) may be configured to provide a notification to the user based on the user's usage of the inhalation device 900. For example, the computer-readable storage medium may be configured to cause the processor of the external device to generate feedback for the user based on data received from the electronics module 120. For example, the computer-readable storage medium may be configured to cause the processor of the external device to generate daily, weekly, or monthly report, provide confirmation of error events or notifications, provide instructive feedback to the user, and/or the like, where the reports, notifications, and/or feedback include the usage parameters, events, and/or measurements associated with the inhalation device 900. The feedback and/or notification may, for example, be the illumination of an LED, the generation of an audible output via a speaker of the inhalation device 900 or the external device 902, the presentation of a message via the display of the external device 902, the presentation of an error video or the instructions for use via the display of the external device 902, by sending a text, email, or instant message to the external device 902, etc.

Further, in some examples, the external device 902 may include a display and software for visually presenting the usage parameters through a GUI on the display. For instance, the external device 902 may be configured to cause the display to present a notification indicating any combination of the events, the measurements, and/or the associated time stamps related to one or more inhalation devices 900. For example, the external device 902 may be configured to generate a GUI that presents any data associated with an inhalation event (e.g., any combination of the mouthpiece cover opening event, the canister actuation event, any errors events, the one or more signals from the sensor indicative of the user's inhalation, and/or the associated timestamps).

The inhalation device 900 and/or the external device 902 may be configured to generate one or more error events based on the data recorded by the electronics module of the inhalation device 900. For example, the inhalation device 900 and/or the external device 902 may be configured to generate an exhalation event when the one or more signals from the sensor indicate an exhalation occurred (e.g., the one or more signals from the sensor indicate a positive pressure change, for instance, when the sensor system comprises a pressure sensor). The inhalation device 900 and/or the external device 902 may be configured to generate an error event (e.g., a mishandling event) in response to the switch contact contacting the second contact pad and the third contact pad of the electronics module and the one or more signals from the sensor indicating measurements below a threshold (e.g., the one or more signals from the sensor indicating pressure measurements below a threshold that is indicative of no inhalation, such as pressure measurements that correspond to a flow rate that is less than 20 L/min). For example, the inhalation device 900 and/or the external device 902 may be configured to generate an error event (e.g., a mishandling event) based on the electronics module recording a canister actuation event but the one or more signals from the sensor indicating measurements below the threshold. Further, in some examples, the inhalation device 900 and/or the external device 902 may be configured to generate a timeout event in response to the electronics module recording a mouthpiece covert opening event, but not a subsequent canister actuation event or the recording of measurements from the sensor system that exceeds a threshold (e.g., 20 L/min) within a set time period, such as 60 seconds.

Further, the inhalation device 900 and/or the external device 902 may be configured to generate a no inhalation error event based on the electronics module of the inhalation device 900 recording a mouthpiece cover opening event and a canister actuation event without the one or more signals from the sensor indicating a user's inhalation (e.g., without the one or more signals from the sensor indicating measurements that are indicative of a user's inhalation, such as measurements that correspond to a flow rate greater than 20 L/min). Also, the inhalation device 900 and/or the external device 902 may be configured to generate a multiple inhalation error event in response to the one or more signals from the sensor system indicating that multiple inhalation occurred during and/or after a single canister actuation event (e.g., where the signals from the sensor system exceeds a threshold, such as 20 L/min, fall below the threshold for a period of time, like 1 second, and then exceed the threshold again). Finally, in some examples, the inhalation device 900 and/or the external device 902 may be configured to generate an excessive inhalation or air vent blockage error event when the one or more signals from the sensor system exceed an upper threshold, such as when the signals correspond to a flow rate that is greater than 300 or 500 L/min).

The DHP 906 may be configured to receive and aggregate data from inhalation devices 900 that are associated with a plurality of different users. For example, the DHP 906 may be configured to receive and store inhalation events, the mouthpiece cover opening event, the canister actuation event, the one or more signals from the sensor indicative of the user's inhalation, error events, associated timestamps, etc. for a plurality of inhalation devices 900 (e.g., inhalation devices 900 of the same and/or different users). The DHP 906 may be configured to analyze and manipulate the data. Further, the DHP 906 may be configured to provide data to the computer 908 associated with a health care provider (e.g., via the dashboard application). The inhaler data may include any of the data described with reference to the inhalation devices 100 described herein.

The inhaler data may be associated with an inhalation device 900 and/or a user profile, for example, at the external device 902 and/or at the DHP 906. One user profile may be associated with one or more inhalation devices 900 of the same and/or different medicament types. The DHP 906 may also de-identify (e.g., unassociate) the inhaler data with a particular user profile, and the DHP 906 may perform analytics on de-identified data relating to the inhalation device 900. Although described as receiving the inhaler data from the external device 902, in some examples, the DHP 906 may receive the data directly from the inhalation devices 900 themselves, such as in instances where the transmission modules on the inhalation devices 900 include cellular chipsets that are capable of communicating directly with the DHP 906.

The DHP 906 may be configured to enable, and in some instances initiate, the transfer of data from the external devices 902. For example, the DHP 906 may expose a REST API to the external devices 902. The DHP 906 may also store incoming data from the computers 908. The DHP 906 may receive the patient's consent via their respective external device 902.

The DHP 906 may include a dashboard application that may be accessible by the computer 908 associated with a health care provider. In some examples, the dashboard application is a web application (e.g., a web portal). For example, the DHP 906 is configured to provide data, such as inhaler data, to clinicians and physicians through the use of the dashboard application (e.g., via a REST API).

The DHP 906 may cause the computer 908 associated with the health care provider to provide inhaler data to practitioners and health care professionals to allow them to view inhaler data specific to a program to which a patient has consented. In one example, the DHP 906 causes the computer 908 associated with the health care provider to provide the inhalation data via a graphical user interface (GUI) that is presented on the health care provider's computer. The GUI may provide specific information for each user, and for example, the information may be aggregated by medicament type for all of the user's inhalation devices 900 that provide a particular medicament type. For instance, the GUI may provide a total number of inhalation events that have occurred over a period of time, such as one week or 30 days, for all inhalation devices 900 that include a particular medicament type, a percentage of the inhalation events that are categorized as good inhalation events, and an all-connected duration that represents the oldest, or least recent, last synchronization time of the inhalation devices 900 of a particular medication type that are associated with the user.

What is claimed is:

1. An inhaler comprising:
a canister elongate along a longitudinal axis, the canister containing medicament;
a movable inner housing that is configured to move with the canister when the canister is moved to administer a dose of medicament;
an electronics module comprising a processor, a power supply, a sensor, and a plurality of contact pads; and
a switch contact affixed to the moveable inner housing;
wherein, in response to movement of the movable inner housing, the switch contact is configured to move from a first position where the switch contact is not contacting the plurality contact pads to a second position where the switch contact is contacting at least one of the plurality of contact pads to change a power state of one or more of the processor and sensor.

2. The inhaler of claim 1, wherein the movable inner housing substantially surrounds the canister.

3. The inhaler of claim wherein the switch contact is affixed to an outer surface of the moveable inner housing.

4. The inhaler of claim 1, wherein the movable inner housing is configured to be in a first configuration when the canister is in a first position, a second configuration spaced from the first configuration along the longitudinal axis when the canister is in a second position, and a third configuration spaced from the first and second configurations along the longitudinal axis when the canister is in a third position, wherein the canister is configured to administer a dose of medicament when moved from the second position to the third position.

5. The inhaler of claim 4, wherein the canister is configured to move from the first position to the second position in response to a movement of a mouthpiece cover of the inhaler to expose a mouthpiece of the inhaler.

6. The inhaler of claim wherein the first configuration comprises a rest configuration in which a metering valve of the canister is in a refill position;
wherein the second configuration comprises a prepared configuration in which the canister is actuatable by user inhalation induced airflow; and
wherein the third configuration comprises an actuation configuration in which the metering valve is in a dose delivery position.

7. (canceled)

8. The inhaler of claim 1, wherein the movable inner housing comprises a force holding unit configured to maintain a force for actuating the canister until a user inhales through the mouthpiece of the inhaler.

9. The inhaler of claim 8, wherein the force holding unit comprises a compression spring, a lower cap that engages the canister, a diaphragm attached to an upper surface of the lower cap, and a pivotally mounted flap valve for selectively sealing a valve port located in the diaphragm.

10. The inhaler of claim 9, wherein an inhalation by a user causes air to pivot the flap valve, which opens a valve channel in the diaphragm allowing passage of the air into a volume between the diaphragm and the lower cap, which produces expansion of the compression spring and a downward motion of the canister, and actuation of a metering valve of the canister to release a measured dose through into a mouthpiece of the inhaler.

11. The inhaler of claim 1, further comprising a main housing that encloses the canister, the electronics module, and the movable inner housing.

12. The inhaler of claim 1, wherein the plurality of contact pads comprises a first contact pad, a second contact pad, and a third contact pad; and
wherein the switch contact comprises a first prong that is configured to contact either the first contact pad or the third contact pad, and a second prong that is configured to contact the second contact pad.

13. The inhaler of claim 12, wherein, when the switch contact is in the first position, the first and second prongs are configured to not be in contact with any of the first, second, or third contact pads, and a metering valve of the canister is configured to be in a refill position;
wherein, when the switch contact is in the second position, the first prong is configured to be in contact with the first contact pad, and the second prong is configured to be in contact with the second contact pad; and
wherein, when the switch contact is in a third position, the first prong is configured to be in contact with the third contact pad, the second prong is configured to be in contact with the second contact pad, and the metering valve of the canister is configured to be in a dose delivery position.

14. The inhaler of claim 13, wherein the second prong of the switch contact is in continuous contact with the second contact pad as the switch contact moves from the second position to the third position.

15. The inhaler of claim 13, wherein, when the switch contact moves into the second position, the processor is configured to record a mouthpiece cover opening event; and
wherein, when the switch contact moves into the third position, the processor is configured to record a canister actuation event.

16. (canceled)

17. (canceled)

18. The inhaler of claim 12, wherein the first contact pad is located above the third contact pad on a printed circuit board (PCB) and adjacent the second contact pad, and wherein the third contact pad is located below the first contact pad on the PCB and adjacent the second contact pad.

19. (canceled)

20. (canceled)

21. The inhaler of claim 1, wherein the plurality of contact pads comprises a first contact pad, a second contact pad, and a third contact pad; and
wherein the switch contact comprises a single prong that is configured to contact the first contact pad and the second contact pad simultaneously, and configured to contact the second contact pad and the third contact pad simultaneously.

22. The inhaler of claim 21, wherein, when the switch contact is in the first position, the single prong is configured to not be in contact with any of the first, second, or third contact pads, and a metering valve of the canister is configured to be in a refill position;
wherein, when the switch contact is in the second position, the single prong is configured to be in contact with the first contact pad and the second contact pad; and
wherein, when the switch contact is in a third position, the single prong is configured to be in contact with the second contact pad and the third contact pad, and the metering valve of the canister is configured to be in a dose delivery position.

23.-29. (canceled)

30. The inhaler of claim 1, wherein, when the switch contact is in the first position, the processor is configured to be in an off state or a sleep state and the sensor is configured to be in an off state;
wherein, when the switch contact is in the second position, the processor is configured to be in an active state and the sensor is configured to be in an active state.

31. The inhaler of claim 1, wherein the electronics module further comprises a printed-circuit board (PCB), wherein the PCB defines a first portion that comprises the processor, the power supply, and the sensor, and a second portion that comprises the contact pads, wherein the first portion is oriented at a substantially perpendicular angle with respect to the second portion.

32. The inhaler of claim 31, wherein the first portion of the PCB resides between a top surface of the canister and a top, interior surface of a main housing of the inhaler, and the second portion of the PCB extends along an inner, sidewall of the main housing.

33.-74. (canceled)
